(19) United States
(12) Patent Application Publication
(10) Pub. No.: US 2008/0050460 A1
(43) Pub. Date: Feb. 28, 2008

(54) Uses of lectin-conotoxin

(76) Inventor: Tor C. Savidge, League City, TX (US)

Correspondence Address: Benjamin Aaron Adler;ADLER & ASSOCIATES, 8011 Candle Lane, Houston, TX, 77071, US

(21) Appl. No.: 11/811,218

(22) Filed: Jun. 8, 2007

Related U.S. Application Data
(60) Provisional application No. 60/811,948, filed on Jun. 8, 2006.

(51) Int. Cl. A61K 36/00; A01H 5/00; A61P 37/00; C07H 5/04; C12N 5/04; C12N 15/00; C07H 21/04; A23L 1/28

(52) U.S. Cl. 424/777; 426/655; 435/320.1; 435/375; 514/62; 536/23.6; 536/55.2; 800/317.4

(57) ABSTRACT

Provided herein are novel onco-fetal carbohydrate motifs present in adenocarcinoma cells. This novel carbohydrate motif is preferentially recognized by tomato fruit lectin-conotoxin. Also provided are methods for inhibiting proliferation and growth of cancer cells and for treating pathophysiological conditions, e.g., cancer, chronic pain, inflammation and/or a microbial infection, by contacting such cells with or administering tomato fruit lectin-conotoxin or similar natural or bioengineered molecules. In addition provided herein are genetically modified plants and seed, fruit, progeny, and hybrids therefrom and a genetically modified foodstuffs overexpressing a lectin-conotoxin. Further provided are DNA and expression vectors expressing lectin-conotoxin described herein.

### CROSS-REFERENCE TO RELATED APPLICATION

This nonprovisional application claims benefit of provisional U.S. Ser. No. 60/811,948, filed Jun. 8, 2006, now abandoned.

### BACKGROUND OF THE INVENTION

1. Field of the Invention

The present invention relates generally to the fields of glycobiology and therapeutics. More specifically, the present invention relates to the use of tomato lectin-conotoxin in the prevention and treatment of cancer, as an anti-inflammatory agent, as an analgesic and as an anti-microbial agent.

2. Description of the Related Art

Increased expression of embryonic or onco-fetal carbohydrate antigens and inappropriate expression of blood group antigens have been detected in many types of human carcinoma using antibodies with well-defined specificities (1). Many of these differential onco-fetal glycosylation patterns appear to be caused by an incomplete O-linked glycan structure (2,3). Another type of carbohydrate alteration is observed in viral or oncogene-transformed rodent fibroblasts where there is an increased branching at the trimannosyl core of complex-type asparagine (N)-linked oligosaccharides, and in particular, increased-GlcNAcβ1-6Man1-6Manβ3 linked antennae (4). It is believed that these β1-6 branched oligosaccharides may contribute directly to the malignant or metastatic phenotype of tumour cells since glycosylation mutants of the metastatic tumour cell line MDAY-D2, which are deficient in β3-6GLcNAc transferase V activity, show loss of metastatic potential but retain full tumorigenic potential (5,6). In addition, swainsonine inhibits in vivo organ colonisation by metastatic MDAY-D2 cells and B16 melanoma cells by specific inhibition of N-linked oligosaccharide processing by blocking the pathway prior to initiation of the β1-6 linked antennae. Several tumour-associated protein antigens are also developmentally regulated. Examples of such onco-fetal proteins include carcinoembryonic antigen, alphafetoprotein, placental alkaline phosphatase and 5T4, a leucine-rich-repeat trophoblast glycoprotein.

Lectins are naturally occurring glycoproteins that bind carbohydrate-residues with great affinity and specificity. They are essential and omnipresent plant constituents. Plant lectins have also been used to detect changes in carbohydrate expression in tumours (3,7). For example, increased Arachis hypogaea binding (i.e. to unsubstituted Galβ1-3GalNAc-O) has been correlated with clinical course in the progression of colorectal carcinoma from ulcerative colitis. Helix pomatia lectin binding (i.e. to GalNAc-O) has been correlated with decreased survival time in breast carcinoma patients. In both of these cases binding sites appear to be of incomplete O-linked glycan structures, while neoexpression of lactosamine-based embryonic structures appears to result from increased expression of transferase activities in carcinomas.

Epidemiological studies have demonstrated that the consumption of tomatoes or tomato-based products has a beneficial effect on a number of health issues, including the prevention of cancer. This has been largely attributed to the presence of the anti-oxidant lycopene in tomatoes. However, a number of independent studies have demonstrated the presence of specific lectins in tomatoes that possess potent biological activity (8,9). The binding of these tomato lectins to glycoproteins expressed by tumors may provide alternative methods for treating such tumors, especially if these lectins inhibit tumor cell growth and proliferation. Binding studies between tomato lectins and tumor glycoproteins can also help in the discovery of novel motifs in tumor glycoproteins that can be targeted for treating specific cancers.

The prior art is deficient in methods that can use the tomato lectin-conotoxin to treat cancer, pain, inflammation and a microbial infection. The present invention fulfills this long-standing need and desire in the art.

### SUMMARY OF THE INVENTION

The present invention is directed to a novel lectin binding carbohydrate motif present on tumor cells. This novel carbohydrate motif comprises extended oligomers of N-acetylglucosamine units, forming a chitobiose carbohydrate structure. A chimeric lectin-conotoxin present in the fruit of the tomato plant preferentially binds this novel motif.

The present invention is further directed to a pharmaceutical composition comprising a lectin-conotoxin that binds this novel carbohydrate motif and a pharmaceutically acceptable vehicle.

The present invention is further directed to inhibiting the growth and proliferation of cancer cells, comprising contacting such cells with the instant pharmaceutical composition. This lectin-conotoxin preferentially binds a novel carbohydrate motif present on cancer cells and suppresses the tyrosine kinase activity and calcium ion entry in such cells causing cell death.

The present invention is also directed to a method for treating a pathophysiological state in an individual, comprising administering the instant composition to the individual. The pathophysiological state can be a cancer, chronic pain, inflammation or a microbial infection. The method can further comprise administering other therapeutic agents to the individual that are generally used to treat cancer, pain/inflammation or microbial infections.

The present invention is also directed to a genetically modified plant. The plant overexpresses a lectin-conotoxin comprising the amino acid sequence of SEQ ID NO:1. The present invention is further directed to a genetically modified food product. The food product comprises excessive amounts of a lectin-conotoxin comprising the amino acid sequence of SEQ ID No: 1.

The present invention is still further directed to an expression vector comprising the nucleic acid sequence encoding a lectin-conotoxin comprising the sequence of SEQ ID NO: 1 and regulatory elements required to express the lectin-conotoxin. The nucleic acid sequence encoding the lectin-conotoxin comprises the sequence of SEQ ID No: 2.

The present invention is further directed to a conotoxin motif having the amino acid sequence of SEQ ID NO: 3. The present invention is further directed to a DNA encoding a protein, wherein the protein has a conotoxin motif and wherein the DNA is selected from the group consisting of: (a) isolated and purified DNA consisting of the sequence of SEQ ID NO: 4; (b) isolated and purified DNA which hybridizes at high stringency conditions to the antisense complement of the isolated DNA of (a) above; and (c) isolated and purified DNA differing from the isolated DNAs of (a) and (b) above in codon sequence due to the degeneracy of the genetic code.

The present invention is also directed to an expression vector capable of expressing the instant DNA. The expression vector comprises regulatory elements necessary for expressing the protein encoded by this DNA. Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others, which will become clear, are attained and can be understood in detail, more particular descriptions of the invention are briefly summarized. The above may be better understood by reference to certain embodiments thereof, which are illustrated in the appended drawings. These drawings form a part of the specification. It is to be noted; however, that the appended drawings illustrate preferred embodiments of the invention and therefore are not to be considered limiting in their scope.

FIGS. 1A-1E show the amino acid sequence and motifs of tomato lectin-conotoxin. FIG. 1A (SEQ ID NO: 1) shows the amino acid sequence of a tomato lectin-conotoxin and FIG. 1B shows its functional motifs. FIG. 1C shows the conotoxin motif (SEQ ID NO: 3) and FIG. 1D (SEQ ID NO: 6) shows the conotoxin+chitin binding motif of the tomato lectin-conotoxin. FIG. 1E shows the tomato lectin-conotoxin (LEL) motif.

FIGS. 2A-2B show the onco-fetal glycosylation pattern recognised by tomato lectin-conotoxin. FIG. 2A shows the glycosylation pattern recognized by tomato lectin-conotoxin in onco-fetal cells, adenocarcinomas and fetal xenograft brush border membrane. FIG. 2B lists the glycosylation pattern recognized by tomato lectin-conotoxin in onco-fetal cells, adenocarcinomas and pediatric intestine with fetal cell xenograft.

FIG. 3 shows tomato lectin-conotoxin slot blots of epithelial microvillus membrane preparations. High lectin binding is seen in fetal (FSI) and xenograft (XSI) small intestine as compared to control pediatric small intestine. Also, elevated binding is seen in inflammatory bowel diseases such as Crohn's disease (CD) and ulcerative colitis (UC) compared with normal colon (N).

FIG. 4 shows confocal fluorescent micrograph of FITC-labeled tomato lectin-conotoxin (green) with intense binding to Caco-2 intestinal adenocarcinoma cells when exposed for 10 min. The cells were pulsed with the tomato lectin-conotoxin for 60 min at 37° C.

FIGS. 5A-5B show flow cytometry fluorescent-lectin binding profiles to Caco-2 (FIG. 5A) and HT29 (FIG. 5B) intestinal cell lines.

FIG. 6 shows inhibition of fluorescent tomato lectin-conotoxin binding to surface N-acetyl-glucosamine on HT29 cells by unlabeled potato lectin (STL) with similar sugar specificity.

FIG. 7 shows inhibition of fluorescent tomato lectin-conotoxin binding to surface N-acetyl-glucosamine on HT29 cells by chitin hydrolysate (rich in N-acetyl-glucosamine oligomers).

FIG. 8 shows surface plasmon resonance demonstrating the kinetics of tomato lectin-conotoxin binding to Caco-2 plasma membrane and detachment by addition of chitin hydrolysate.

FIGS. 9A-9B show inhibition of tyrosine kinase activity by tomato lectin-conotoxin (LEL) in Caco-2 (FIG. 9A) and HT-29 (FIG. 9B) intestinal cells compared with control non-binding lectin (DBA) and positive control calcium ionophores (ION/A23187).

FIG. 10 shows that intestinal adenocarcinoma Caco-2 cell proliferation is significantly inhibited at a 100 nM concentration of tomato lectin-conotoxin as compared to non-binding DBA lectin (*, p<0.05).

FIG. 11 shows MTT assay measuring cellular mitochondrial function demonstrating cytotoxicity of tomato lectin-conotoxin (LEL) compared with nonbinding lectin DBA.

FIG. 12 shows increased side-scatter in flow cytometry of HT29 cells post treatment with tomato lectin-conotoxin compared with nonbinding lectin BSL-1, indicative of membrane disruption associated with apoptosis. Calcium ionophores also induce membrane disruption associated with apoptosis (ION/A23187).

FIG. 13A-13C show flow cytometry analysis HT29 cells expressing surface phosphatidyl serine (recognised by fluorescent annexin). FIG. 13A shows the base level of phosphatidyl serine, FIG. 13B shows the increase in level of phosphatidyl serine as a result of apoptosis by tomato lectin-conotoxin (LEL) and FIG. 13C shows the increase in level of phosphatidyl serine as a result of inflammatory mediators (tumour necrosis factor-alpha/interferon gamma (TNFα/IFNγ).

FIG. 14 shows upregulation of a key apoptotic protease (caspase 3/CPP32) as a result of apoptosis following tomato lectin-conotoxin or inflammatory mediator exposure (FAS/interferon gamma or tumour necrosis factorα/interferon gamma).

FIG. 15 shows elevations of enterocyte line brush border enzymes such as alkaline phosphatase (AP), dipeptidyl peptidase (DPPIV), gamma-glutamyl transferase (GTT), which is indicative of enhanced cellular differentiation proliferation post tomato lectin-conotoxin treatment compared with non-binding lectin control (DB3A).

FIG. 16 shows elevated immunofluorescence of dipeptidyl peptidase (DPPIV/CD26) in epithelial cell lines following tomato lectin-conotoxin exposure compared with control non-binding lectin (BSL-1) or tumour necrosis factor/interferon gamma (TNFα/IFNγ).

FIGS. 17A-17B show the hypothetical tomato lectin-conotoxin-gp96 interaction. FIG. 17A shows that the initial interaction of gp96 and tomato lectin-conotoxin is through (1) lectin-activity, and (2) a potential irreversible extensin-like activity. Extensin-like activity could also modify cellular function independently. FIG. 17B shows that the result of such interactions is a loss of gp96 chaperone function (green-important chaperoned cancer survival co-factors).

FIGS. 18A-18B show the hypothetical tomato lectin-conotoxin interaction with ion channels. An initial interaction of ion channels and tomato lectin-conotoxin (LEL) is through lectin-activity, and a potential interaction of the tomato lectin-conotoxin motif with membrane ion channels, for example L-type calcium channels (FIG. 18A). The result of such interactions is a loss of ion entry (e.g. calcium into the cell or from intracellular stores) that results in decreased proliferation and cell death (FIG. 18B).

FIG. 19 shows inhibition of calcium entry in mEGC glial cells by tomato lectin-conotoxin (LEL) which is compared to the calcium entry in the presence of DBA control lectin at a similar concentration. The calcium entry into the mEGC glial cells was measured following stimulation of voltage-gated channels by the addition of 60 mM KCl. The calcium variations at the single-cell level were monitored using a Nikon Diaphot inverted microscope, equipped with a Nikon 40× (1.3 N.A.) oil immersion objective, coupled to a dual monochrometer system via a fiberoptic cable. Fura-2 intracellular fluorescence was measured at an emission wavelength of 510 nm by alternating the excitation wavelength between 340 and 380 nm. Full ratio images were obtained at 1 image per 1.5 seconds. Images were processed using ImageMaster software (PTI).

FIG. 20 shows the death of Caco-2 cells by in vitro expression of the tomato lectin-conotoxin gene in these cells. The LEL gene was expressed in Caco-2 cells using the Vivid Colors™ pcDNA™ 6.2/EmGFP-Bsd/V5-DEST vector from Invitrogen (Carlsbad, Calif.). S-5 and S-20 are sense constructs of the LEL gene; As-7 is the anti-sense construct; C is the empty plasmid; Am C is a positive control plasmid; and A and AS shows that killing of Caco-2 cells is prevented by cotransfection of sense and anti-sense constructs in each cell.

FIGS. 21A-21C show the inhibition of intracellular calcium clearance in HEK cells by the tomato lectin-conotoxin, LEL. The calcium assay is the same as that described for FIG. 19. FIG. 21A shows the intracellular calcium clearance in response to 1 nM gastrin. FIG. 21B shows the delay in intracellular calcium clearance in response to 1 nM gastrin by HEK cells treated with LEL (100 nM for 20 min). FIG. 21C shows that HEK cells treated with LEL (100 nM for 5 min) show delay in clearance of intracellular calcium even after the cells are treated with carbachol (10 mM).

FIG. 22 shows the nucleic acid sequence that encodes for the tomato lectin-contoxin, LEL (SEQ ID NO: 2).

FIGS. 23A-23B show 2D-proteomic gel analysis (FIG. 23A) data that tomato lectin conotoxin induces an endoplasmic reticular stress response with down-regulated gp96. FIG. 23B lists the proteins (downregulated and upregulated) by tomato lectin conotoxin in Caco-2 cells.

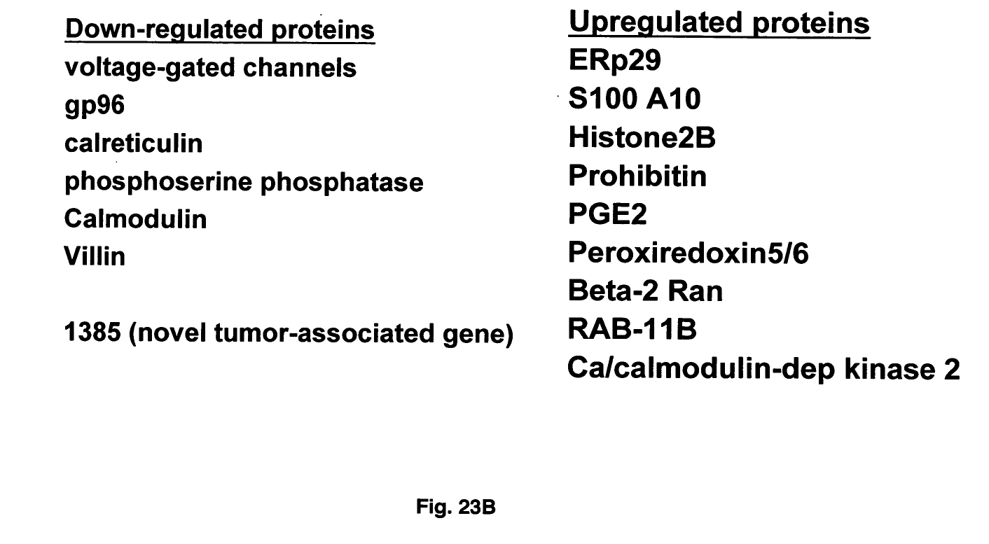

FIG. 24 shows an affymetrix Gene chip array showing significant alterations in specific calcium signaling pathways following treatment with tomato lectin conotoxin.

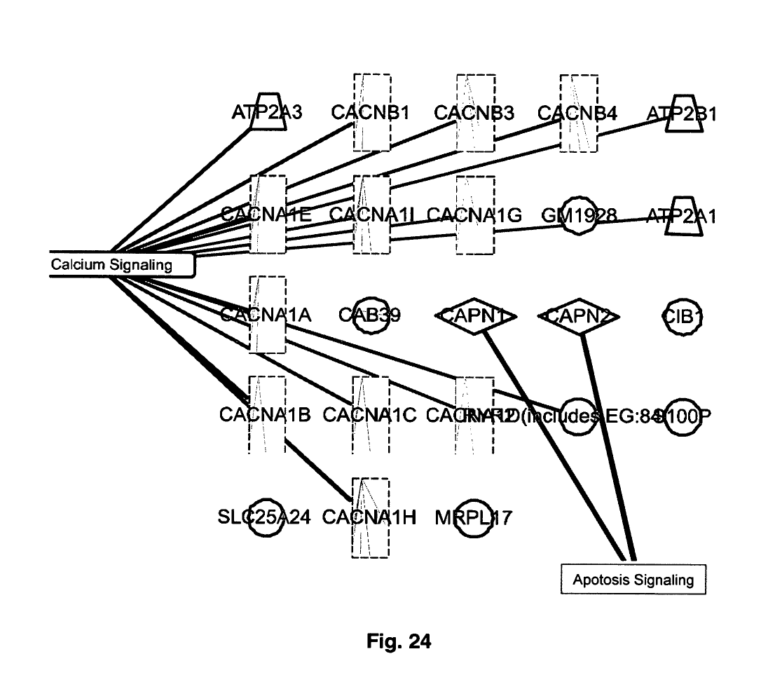

FIG. 25 shows inhibition of voltage gated calcium entry in PC12 neuronal cells.

FIG. 26 shows inhibition of store-operated calcium entry in PC3 prostate cancer cells.

### DETAILED DESCRIPTION OF THE INVENTION

A onco-fetal lectin binding carbohydrate motif present on adenocarcinoma cells was found to preferentially bind a chimeric lectin-conotoxin present in tomato fruit. On binding to adenocarcinoma cells, the lectin-conotoxin suppresses tyrosine kinase activity in these cells, possibly by inhibiting ion channel activity or gp96 function. This lectin-conotoxin was also found to induce apoptosis of adenocarcinoma cells. Accordingly this invention is directed to a novel carbohydrate motif present on tumor cells and the use of a lectin-conotoxin that specifically binds this novel motif to inhibit growth and proliferation of such cells. The invention is also directed to the use of the lectin-conotoxin as an analgesic, anti-inflammatory agent or an anti-microbial agent as the lectin-conotoxin can inhibit ion channel activity.

As used herein “motif” refers to a distinct sequence or pattern of structural units such as amino acids or sugar residues. In proteins, a motif refers to a specific sequence of amino acids, which is associated with a specific structure and/or function. For example an extensin like motif refers to multiple Ser(Pro)n repeats.

As used herein “carbohydrate motif” refers to the specific type and pattern in which sugar residues are arranged to form the carbohydrate unit linked to a glycoprotein or a glycolipid. For example, during the synthesis of a glycoprotein, molecules such as lactose, mannose and glucosamine oligomers are added to the protein structure and this carbohydrate unit is modified during and following protein synthesis to form a final motif that is distinct for each glycoprotein. The sugar oligomer or carbohydrate is generally attached to the protein via asparagines, hydroxylysine, hydroxyproline, serine or threonine. These carbohydrate residues on glycoproteins have functional consequences such as signaling, receptor function, etc.

As used herein “domain” refers to a structurally and functionally defined protein region. In proteins with multiple domains, the combination of the domains determines the function of the protein. For example the Ca⁺²/calmodulin dependent protein kinase has a Ca⁺² binding domain and a separate calmodulin binding domain, which are both required for proper function of the protein.

As used herein ‘chimeric lectin’ refers to a lectin comprising structurally and functionally different domains. For example, the tomato fruit lectin-conotoxin is a chimeric lectin as it comprises several chitin-binding domains that are linked by an extension-like domain and a conotoxin domain. Extensin like domain refers to the motif found in cell wall associated extensions and comprise multiple Ser(Pro)ₙ repeats. Conotoxin motif generally comprises a family of inhibitory cysteine-knot (ICK) like sequences that serve as potent inhibitors for a variety of ion channel activities.

As used herein “onco-fetal” refers to substances associated with tumor formation and present in normal fetal tissue. For example an onco-fetal carbohydrate motif refers to a carbohydrate motif seen in tumor cell glycoproteins and also expressed in normal fetal tissue.

As used herein “chitobiose” refers to a disaccharide formed by two molecules of N-acetyl-D-glucosamine.

As defined herein “genetically modified food product or genetically modified plant” refers to a plant or food product in which the normal DNA is altered by human intervention. For example U.S. Pat. No. 7,034,203 describes a genetically modified tomato that has increased levels of flavonols as compared to the wild type tomato fruit. For example U.S. Pat. No. 6,713,662 discloses genetically modified milk that contains collagen.

As used herein “overexpress” refers to the expression of a compound by an organism at levels higher than that, which is normally expressed by the organism. For example U.S. Pat. No. 7,049,485 decribes transgenic plants containing ligninase and cellulase which degrades lignin and cellulose to fermentable sugars.

As used herein “excessive amounts” refers to the presence of a compound in a food material at levels higher than that, which is normally present in the food material. For example U.S. Pat. No. 7,034,203 describes a genetically modified tomato that has increased levels of flavonols as compared to the wild type tomato fruit.

As used herein “substantially pure DNA” refers to DNA that is not part of the milieu in which the DNA naturally occurs, by virtue of separation of some or all of the molecules of that milieu, or by virtue of alteration of sequences that flank the claimed DNA. The term therefore includes, for example, a recombinant DNA which is incorporated into a vector, into an autonomously replicating plasmid or virus, or into the genomic DNA of a prokaryote or eukaryote; or which exists as a separate molecule (e.g., a cDNA or a genomic or cDNA fragment produced by polymerase chain reaction (PCR) or restriction endonuclease digestion) independent of other sequences. It also includes a recombinant DNA, which is part of a hybrid gene encoding additional polypeptide sequence, e.g., a fusion protein.

By “high stringency” is meant DNA hybridization and wash conditions characterized by high temperature and low salt concentration, e.g., wash conditions of 65° C. at a salt concentration of approximately 0.1×SSC, or the functional equivalent thereof. For example, high stringency conditions may include hybridization at about 42° C. in the presence of about 50% formamide; a first wash at about 65° C. with about 2×SSC containing 1% SDS; followed by a second wash at about 65° C. with about 0.1×SSC.

As used herein, the term “contacting” refers to any suitable method of bringing the instant pharmaceutical composition in contact with the cell such that the lectin-contoxin can exert its effect. In vitro or ex vivo this is achieved by exposing the cells to the compound in a suitable medium. The cells can also be transfected with instant expression vectors that express a lectin-conotoxin. The vector then expresses the lectin-conotoxin within the cell and thereby exerts the desired effect. For in vivo applications, any known method of administration is suitable as described infra.

In one embodiment the present invention is directed to a novel carbohydrate motif. The carbohydrate motif comprises extended oligomers of N-acetyl glucosamine forming a chitobiose carbohydrate unit. Generally this carbohydrate motif is seen in glycoproteins of cancer cells. Specifically the cancer is intestinal adenocarcinoma, prostate adenocarciona, renal adenocarcinoma, melanoma, lymphomas or gliomas. The novel carbohydrate motif is recognized by tomato lectin-conotoxin. In one aspect, the lectin is from the tomato species Lycopersicum esculentum. This lectin is commonly known as Lycopersicum esculentum lectin (LEL).

The binding specificity of tomato lectin-conotoxin is directed at tri- or more-highly branched complex-type-N-glycans containing different sugar chains. N-acetyllactosamine structure is the primary binding site of tomato lectin-conotoxin for complex-type N-glycans. The chitobiose core is the primary binding site for high mannose-type N-glycans. This chitobiose carbohydrate structure is not freely accessible to tomato lectin-conotoxin in complex-type-N-glycans, and becomes a novel tumor cell target for tomato lectin-conotoxin as these cells are undifferentiated and possess less complex sugar chains.

In another embodiment is provided a pharmaceutical composition comprising a lectin-conotoxin that binds the carbohydrate motif described supra and a pharmaceutically acceptable vehicle. In all aspects of this embodiment the lectin-conotoxin is obtained from a natural source or is a bioengineered molecule. Natural sources of the lectin-conotoxin can be any plant, fruit, seeds, leaves and such materials. More preferably the lectin-conotoxin is purified from the fruit of Lycopersicum esculentum. The lectin-conotoxin can also be obtained by expressing the lectin-conotoxin using expression vectors in an organism such as a plant or an animal or in a plant or animal cell culture.

In all aspects of this embodiment the lectin-conotoxin can comprise one or more chitin binding domain(s) separated by an extensin and a conotoxin domain. Preferably the lectin-conotoxin has two chitin binding domains separated by an extensin domain and a conotoxin domain. Most preferably the lectin-conotoxin comprises the amino acid sequence of SEQ ID NO: 1. Furthermore, in all aspects of this embodiment the lectin-conotoxin binds gp96 heat shock protein or ion channels in a cell and suppresses tyrosine kinase activity or ion channel activity in a cell. Specifically, the ion channels bound by the lectin-conotoxin are the voltage-gated, store-operated and transient receptor potential (TRP) calcium channels. Moreover, the binding of the lectin-conotoxin to gp96 is effective to downregulate or to suppress gp96. The downregulation or suppression of gp96 is effective to induce an endoplasmic reticulum stress response.

In another embodiment, the present invention is directed to a method of retarding growth and proliferation of cancer cells, comprising contacting the cancer cells with the composition described supra. Specifically the cancer cells that can be suppressed by this method are adenocarcinoma, melanoma, lymphoma and glioma cells. A representative example of an adenocarcinoma is intestinal or prostate adenocarcinoma. In all aspects of this embodiment the lectin-conotoxin, source of lectin-conotoxin and the mechanism by which the lectin-conotoxin exerts its antineoplastic effect are as described supra.

The tomato fruit lectin-conotoxin has subunits that are connected by a linker that comprises both extensin-like and conotoxin-like motifs. A person having ordinary skill in the art could easily substitute the tomato lectin-conotoxin with other plant lectins, which have extensin- and conotoxin-like motifs and can bind the novel carbohydrate motif described supra. Furthermore bioengineered chitin-binding like molecules, which also comprise both extensin and/or conotoxin like motifs can also be used to practice this method.

In another embodiment is provided a method to treat a pathophysiological state in an individual, comprising administering to the individual the pharmaceutical composition described supra. In all aspects of this embodiment the lectin-conotoxin, source of lectin-conotoxin and the mechanism by which the lectin-conotoxin exerts its antineoplastic effect are as described supra. In a related aspect, the lectin conotoxin inhibits growth and proliferation of premalignant and neoplastic cells thereby reducing the risk of cancer in an individual. In all aspects of this embodiment the pathophysiological state is a cancer, chronic pain, inflammation or a microbial infection. Further, in all aspects, the cancer can be intestinal adenocarcinoma, renal adenocarcinoma, melanoma, lymphoma or glioma.

In a related embodiment, the method can further comprise administering an anticancer agent, an analgesic/anti-inflammatory agent and/or an anti-microbial agent to combat cancer, pain/inflammation or a microbial infection in an individual. In all aspects of this embodiment a representative anticancer agent can be a chemotherapeutic agent such as for example, methotrexate or mercaptopurine or a radiotherapeutic agent such as such as for example, ¹³¹I-metaiodobenzulguanidine (MIBG) or ⁹⁰Y-DOTA-D-Phel-Tyr3-octreotide (DOTATOC). In all aspects of this embodiment representative analgesic/anti-inflammatory agents are aspirin, paracetamol, ibuprofen, ketoprofen, naproxen sodium, diflunisal, indomethacin, sulindac, corticosteroids etc. Further, in all aspects of this embodiment, representative antimicrobial agents can be any antibiotic or antifungal agent that is generally used to treat such infections.

In yet another embodiment, the invention is directed to a genetically modified plant that overexpresses a lectin conotoxin comprising the sequence of SEQ ID NO: 1. The lectin-conotoxin can be expressed in any part of a plant such as for example the leaf or the fruit. Specifically the genetically modified plant is a tomato plant.

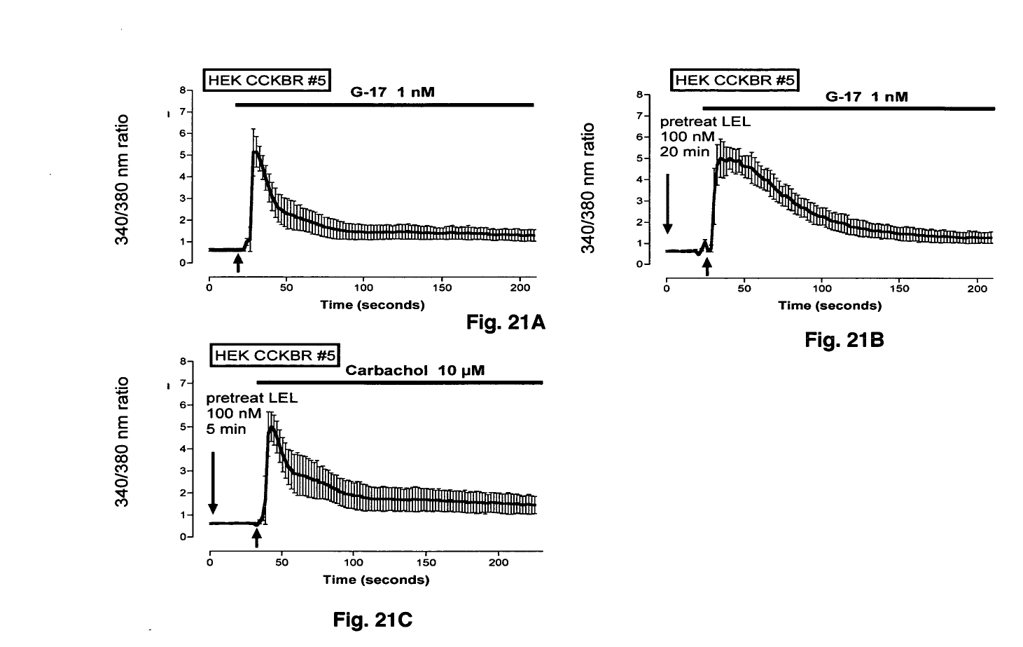

In yet another embodiment is provided a food material that comprises excessive amounts of a lectin-contoxin comprising the sequence of SEQ ID NO: 1. For example the food material can be a fruit, a seed, a vegetable, an egg, milk or meat. In yet another embodiment is provided an expression vector that comprises the nucleic acid sequence encoding a lectin-conotoxin comprising the sequence of SEQ ID NO: 1 and regulatory elements required to express the lectin-conotoxin. The nucleic acid encoding the lectin-conotoxin comprises the sequence of SEQ ID NO: 2. Generally the expression vector can be a plasmid, a phage or any other vectors optimized for expression of plant proteins. An example of an expression vector that may be used to clone the LEL gene is the Vivid Colors™ pcDNA™ 6.2/EmGFP-Bsd/V5-DEST vector from Invitrogen (Carlsbad, Calif.). The expression vector can be used to produce the lectin-contoxin in an organism such as a plant or animal. The lectin-conotoxin thus produced can be purified and used in the instant pharmaceutical composition. The expression vector may also be directly administered to an individual to treat a pathophysiological state such as cancer, pain, inflammation or a microbial infection. The expression vector can also be used to transfect tumor cells (FIG. 20).

Methods which are well known to those skilled in the art can be used to construct expression vectors containing appropriate transcriptional and translational control signals. See for example, the techniques described in Sambrook et al., 1989, Molecular Cloning: A Laboratory Manual (2nd Ed.), Cold Spring Harbor Press, N.Y. A gene and its transcription control sequences are defined as being “operably linked” if the transcription control sequences effectively control the transcription of the gene.

In yet another embodiment is provided a contoxin motif comprising the sequence of SEQ ID NO: 3. Generally this conotoxin motif is tomato fruit lectin-contoxin. The conotoxin motif can inhibit ion channel activity. It is contemplated that peptides comprising the instant conotoxin motif can be used as analgesic/anti-inflammatory or antimicrobial agent in view of the inhibitory effect of known conotoxin motifs on ion channel activity. A peptide comprising the conotoxin motif may also be directly expressed in target cells to alleviate pain/inflammation or to combat a microbial infection in an individual. The nucleotide sequences of SEQ ID NOS: 4 can be used to clone the conotoxin motif containing peptide of the tomato lectin-conotoxin in an expression vector. An example of an expression vector that may be used to express the contoxin motif containing peptide is the Vivid Colors™ pcDNA™ 6.2/EmGFP-Bsd/V5-DEST vector from Invitrogen (Carlsbad, Calif.).

In another embodiment is provided a DNA encoding a protein, wherein the protein has a conotoxin motif and wherein the DNA is (a) isolated and purified DNA consisting of the sequence of SEQ ID NO: 4; (b) isolated and purified DNA which hybridizes at high stringency conditions to the antisense complement of the isolated DNA of (a) above; and (c) isolated and purified DNA differing from the isolated DNAs of (a) and (b) above in codon sequence due to the degeneracy of the genetic code.

In yet another embodiment is provided an expression vector capable of expressing the instant DNA. The expression vector comprises regulatory elements necessary for expressing the protein encoded by this DNA.

The tomato lectin-conotoxin from Lycopersicum esculentum was found to bind the glycoprotein gp96 in adenocarcinoma cells. gp96 is an inducible homolog of the heat shock protein 90 (HSP90). This protein is greatly upregulated during malignant transformations and contains five potential N-glycosylation sites that are differentially glycosylated in malignant cells. This allows for potent binding of tomato lectin-conotoxins to adenocarcinoma cells. Furthermore this protein was found to chaperone important regulators of tumor cell proliferation such as ZNF225, proliferation cell nuclear antigen (PCNA) and B23 nucleophosmin. In view of these findings it is contemplated that dysfunction of gp96 due to binding of tomato lectin-conotoxin is partly responsible for inhibition of tumor cell proliferation and death.

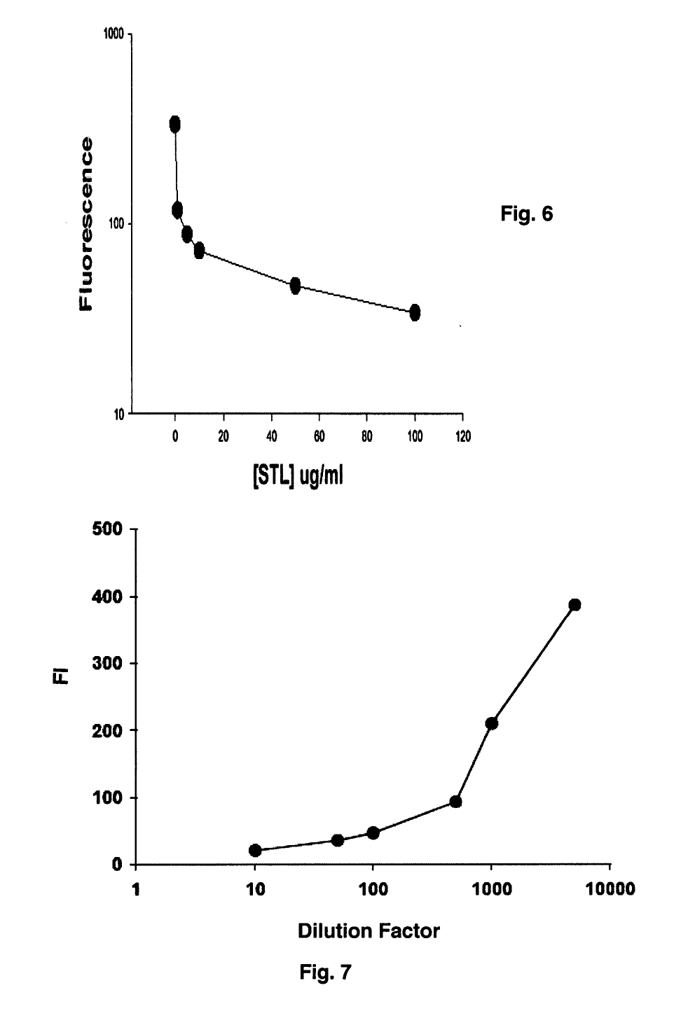

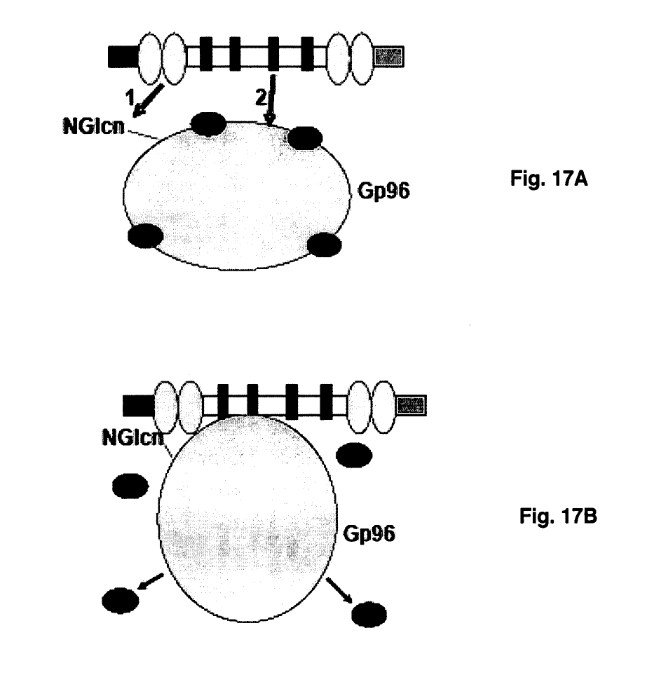

Tomato lectin-conotoxin is a chimeric protein consisting of homologous chitin-binding modules, separated by an extensin-like linker and a conotoxin-like motif. It is contemplated that these independent chimeric functions may act in concert to inactivate molecules such as gp96 on tumour cells, most likely by inhibiting gpP96 mediated protein conformation/refolding resulting in a depletion of oncogenic kinases through proteosomal degradation of immature protein. This notion is supported by the fact that the present invention demonstrates that tyrosine kinase signaling is significantly suppressed in tomato lectin-conotoxin treated tumour cells (FIG. 9A). The unique glycosylation pattern of gp96 can promote initial binding to dietary tomato lectin-conotoxin and the additional extensin-like domain function of the tomato-lectin can then subsequently bind other amino acids and inactivate gp96 function, resulting in altered cellular physiology and death (FIGS. 17A-17B).

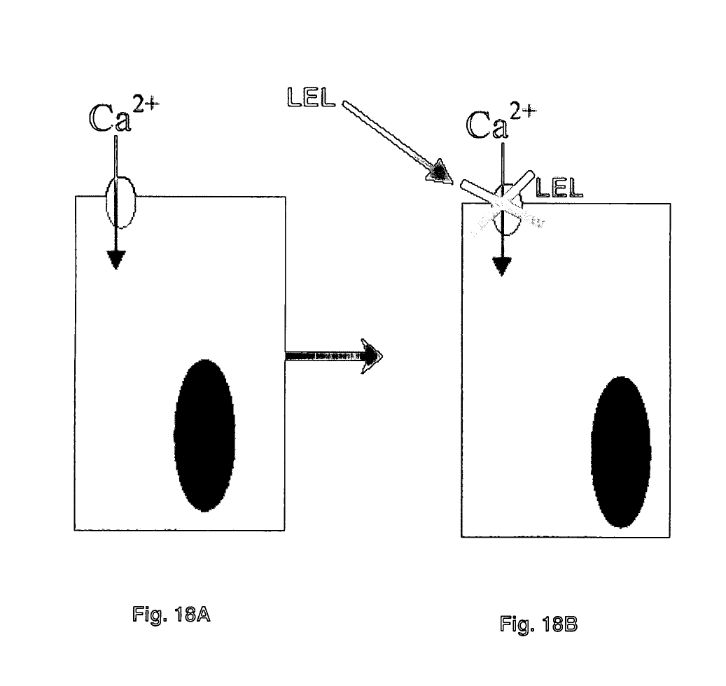

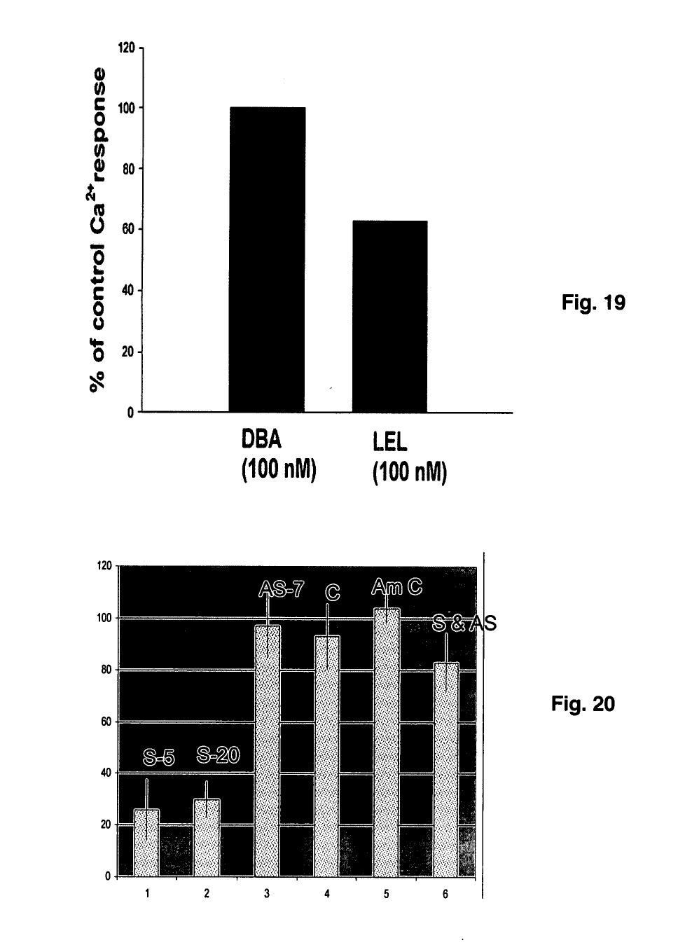

The conotoxin-like motif in tomato lectin-conotoxin may also inhibit ion channel activity on tumor cells that suppresses proliferation and cell survival. This may involve an initial lectin binding event to tumor cell membranes and a subsequent interaction with ion channels, for example the L-type, storage-operated and transient receptor potential calcium channels that are important for tumor cell survival (FIGS. 18A-18B and 19). These calcium channels are up regulated in tumor cells and inhibition using specific chemical antagonists or ω-conotoxins slows tumor cell proliferation and survival.

The instant pharmaceutical composition can be administered by any suitable means, for example, orally, as a tumor preventative daily constituent of the diet, in the form of tablets, capsules, granules or powders; sublingually; bucally; parenterally, such as by subcutaneous, intravenous, intramuscular, or intrasternal injection or infusion techniques (e.g., as sterile injectable aqueous or non-aqueous solutions or suspensions); in dosage unit formulations containing non-toxic, pharmaceutically acceptable vehicles or diluents. The lectin-conotoxin can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The lectin-conotoxin can also be administered liposomally. The tomato lectin-conotoxin can also be administered using the instant expression vectors that express the conotoxin.

Representative examples of oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. Molded tablets, compressed tablets or freeze-dried tablets are exemplary forms, which may be used. Exemplary compositions include formulations with fast dissolving diluents such as mannitol, lactose, sucrose and/or cyclodextrins. Also included in such formulations may be high molecular weight excipients such as celluloses (avicel) or polyethylene glycols (PEG). Preventative treatment may also constitute a daily ingestion of foods/drinks that contain natural or genetically engineered tomato lectin-conotoxin.

Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

The following examples are to illustrate various embodiments of the invention and are not meant to limit the present invention. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Tissue Culture

HT-29 human colon epithelial cells (ATCC HTB38) and Caco-2 human ileocaecal epithelial cells (ATCC HTB37) were grown in DMEM cell culture media containing 10% (v/v) heat inactivated fetal calf serum and contained antibiotics (penicillin/streptomycin (1:1), 100 μg/ml). Cells were incubated at 37° C. in a 5% CO/95% air atmosphere.

### EXAMPLE 2

### MTT Cytotoxicity and Proliferation Assay

In order to determine in vitro lectin-mediated cytotoxic effects on the adherent adenocarcinoma cells lines, the tetrazoium salt 3-[4,5-Dimethylthiazol-2-]-2,5-diphenyltetrazolium bromide (MTT) assay was used. Cells were seeded in 12 well tissue culture plates (Nagle Nunc International) at a concentration of 5×10⁴ cells/ml of medium and lectins were added 24 hours later in serum-free medium. After 48 hours cells were washed 3 times with sterile PBS and the medium (without phenol red) replaced contains 5 mg/ml MTT for 3 hours at 37° C. The insoluble formazan salt was dissolved by the addition of 0.5 ml of 20% (w/v) SDS in 10 mM HCl, followed by overnight (o/n) incubation at 37° C. The absorbance of the converted dye was measured at 550 nm.

### EXAMPLE 3

### Morphological Assessment

Cells undergoing apoptosis were identified by staining monolayers with the DNA dye Hoechst 33258 (5 μg/ml) or by staining adherent and non-adherent cells with acridinine orange and eithidium bromide. For the latter, adherent cells were detached using 0.25% trypsin/0.25% EDTA in PBS for 3-5 minutes, washed, pooled with non-adherent cells, and adjusted to 5×10⁵ cells/ml. 100 μl of a mixture of 100 μg/ml each of acridine orange and ethidium bromide was added to 5×10⁵ cells in a 1 ml volume. Cell preparations were examined by epifluorescence microscopy and flow cytometry.

### EXAMPLE 4

### FITC-Labelled Annexin-V and Propidium Iodide Staining

FITC-conjugated annexin-V (which binds to phoshatidylserine), and propidium iodide were added to 1×10⁵ cells, after which cells were incubated for 15 minutes at room temperature in the dark according to the manufacturer's instructions (Boehringer Mannheim), and cells were analysed by flow cytometry. Early apoptotic cells stain with annexin-V alone, whereas necrotic cells and late apopototic cells stain with both annexin V and propidium iodide.

### EXAMPLE 5

### Activation of Caspase-3

Activation of caspase-3 (CPP32) was determined by detection of the chromophore p-nitroanilide (pNA) after cleavage from the labeled substrate DEVD-pNA (ApoAlert CPP32 Colorimetric Assay, Clontech). Briefly, adherent colon epithelial cells were detached from tissue culture plates, after which 2×10⁶ cells were lysed, and incubated with DEVD-pNA for 1 hour at 37° C. Optical density was measured at 405 nm. HT29 colon epithelial cells incubated with TNFα (20 ng/ml) or anti-Fas (1 μg/ml; Pharmingen) monoclonal antibody, in the presence of IFN-γ (100 U/ml) were used as a positive control for caspase-3 activation. Control reactions included the CPP32 inhibitor DEVD-fmk added prior to the addition of DEVD-pNA. This inhibitor completely blocks caspase-3 activation.

### EXAMPLE 6

### DNA Fragmentation Assay

The ApoAlert DNA fragmentation assay was used according to the manufacturer's recommendation (Clontech) and is based on the terminal deoxynucleotidyl transferase-mediated dUTP nick-end-labelling (TUNEL) method of Gavrieli et al (1992). Terminal deoxynucleotidyl transferase (TdT) catalyses incorporation of fluorescein labeled dUTP at the free 3′-hydroxyl ends of fragmented DNA. The fluorescein labeled DNA can then be quantified using either fluoresence microscopy or flow cytometry. Preincubation of cells for 1 hour at 37° C. with DNAse I (1 μg/ml in DNAse buffer) served as a positive control.

### EXAMPLE 7

### Protein Kinase C Assay

An enzyme-linked immunosorbent assay that utilizes a synthetic Protein Kinase C(PKC) pseudosubstrate and a monoclonal antibody that recognises the phosphorylated form of the peptide was used to measure PKC activity as specified in the manufacturer's instructions (Oncogene Research Products, Calbiochem). Briefly, 10⁷ adenocarcinoma cells were left in serum-free media overnight and incubated with purified tomato lectin-conotoxin (100 nM in serum free media) for 30 minutes at 37° C. Harvested cells were washed in PBS containing 0.2 mM Na₃VO₄, suspended in 1 ml cold sample buffer [50 mM Tris-HCl (pH 7.5), 5 mM EDTA, 10 mM EGTA, 50 mM 3-mercaptoethanol, 1 mM PMSF, 10 mM Benamidine), and sonicated for 30 seconds on ice. Samples were then centrifuged at 100,000 g for 60 min at 4° C. and supernatants collected for measurement of PKC activity and protein determination. 100 μl PKC reaction mixture [25 mM Tris-HCl (pH 7.0), 3 mM MgCl₂, 0.1 mM ATP, 2 mM cAMP, 2 mM CaCl₂, 50 μg/ml phospatidylserine, 0.5 mM EDTA, 1 mM EGTA, 5 mM β-mercaptoethanol] was placed in each test well of a polyvinal plate not coated with the pseudosubstrate and preincubated at 25° C. for 5 minutes. 12 μl of kinase sample was then added to each well and mixed well. 100 μl of reaction mixture was then transferred to each pseudosubstrate-coated well and incubated at 25° C. for 15 minutes after which 100 μl stop solution was added. After washing 5 times with wash solution, 100 μl of biotinylated antibody 2B9 was added to each well and incubated for 1 hour at 25° C. After a repeated washing step, 100 μl of peroxidase-conjugated streptavidin was added to each well and incubated for 1 hour at 25° C. After repeating the washing stage, 100 μl of substrate solution was added to each well and incubated at 25° C. for 5 minutes. Wells were then read at 492 nm after stopping the reaction with 100 μl stop solution.

### EXAMPLE 8

### Protein Tyrosine Kinase Assay

Cells were treated in a similar fashion as described for the PKC assay, although cell lysates were prepared using an extraction buffer containing 20 mM Tris (pH 7.4), 50 mM NaCl, 1 mM EDTA, 1 mM EGTA, 0.2 mM PMSF, 1 μg/ml pepstatin, 0.5 μg leupeptin, 0.2 mM Na₃VO₄, 5 mM mercaptoethanol, with homogenisation followed by sonication for 30 seconds on ice. The lysates were then analysed for tyrosine kinase activity using the method specific by the manufacturer (Oncogene Research Products, Calbiochem). Briefly, kinase reaction buffer was prepared by adding 0.1 mM ATP, 5 mM 2-mercaptoethanol and 2 mM. Na₃VO₄. 10 μL of lysate was added to 90 μl of reaction buffer (in wells containing an appropriate pseudosubstrate) and incubated for 30 minutes at 25° C. After washing the wells with 1× wash buffer, 100 μl of PY20 antibody (1:200 dilution; horseradish peroxidase conjugated) was added for 1 hour at 25° C. After washing, 1001 of substrate solution was added for 6 minutes (in the dark; 25° C.), the reaction stopped with 100 μl stop solution and the absorbance was measured at a dual wavelength of 450/550 nm.

### EXAMPLE 9

### Enzyme Activities

Cells (1×10⁷) were pelleted at 1200 rpm for 5 minutes and sonicated in ice-cold physiological saline using an ultrasonic probe, fractionated in 100 μl aliquots and refrozen at −40° C. until further analysis. The total activity of alkaline phosphatase (EC 3.1.3.1) was determined according to Babson and Read (1959), using the following modifications; 2-amino-2-methyl propan-1-ol (0.25 M, pH 10.4; 5 mM MgCl₂) and β-naphtyl phosphate (4 mM) were used as buffer and substrate, respectively. Cell homogenates were incubated at 30° C. for 30 minutes. The reaction was stopped by adding 0.1 M sodium citrate (pH 5.2). A diazoic reaction was performed with 30 mM o-dianisine tetrazotized for 3 minutes at 25° C. and stopped with 5% trichloroacetic acid. The coloured product of the diazoic reaction was extracted with ethyl acetate and the absorbance measured at 530 nm using β-napthol as the standard. Dipeptidyl peptidase IV (DPPIV) activity was determined at 405 nm using 5.6 mM glycyl-L-prolin-4-nitroanilide.HCl as the substrate in 0.1 M Tris buffer (pH 8.5), after a 15 minute incubation at 37° C. DPPIV immunoreactivity was also determined using flow cytometric analysis of 1×10⁶ cells incubated with 1 μg/ml CD26 monoclonal antibody (Becton-Dickinson).

### EXAMPLE 10

### Statistics

All statistical analyses were calculated using MINITAB statistical software (Minitab Inc.). The Welsh test was adopted to account for non-pooled variance within each population analyzed. Deviations from the null-hypothesis were additionally confirmed using the non-parametric Mann-Whitney U-test for ranks (s.e.m).

### EXAMPLE 11

### Structural-Functional Motifs for Tomato Lectin

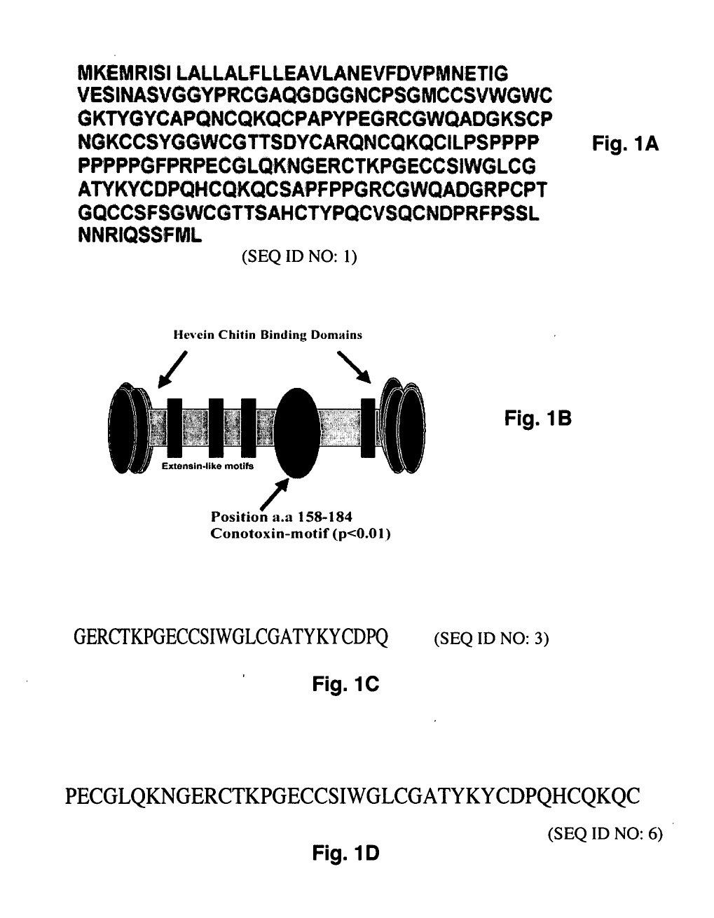

The amino acid sequence and schematic-functional motif structure for tomato lectin (as predicted from its nucleotide sequence) is shown in FIGS. 1A-1B. Tomato lectin is a chimeric protein consisting of homologous chitin-binding modules, separated by an extensin-like linker and a conotoxin-like motif. The extensin-like repeats are hydroxyproline-rich glycoprotein domains that have been implicated in pathogen host defense and in the bioassembly of extracellular matrix. Several proposed functions have been suggested for the extensins, including adhesion and cross-linking of proteins and binding of hydrophobic ligands. A conotoxin-like domain is also contained within this linker, and may also possess important biological and anti-tumour activity. The conotoxin superfamily comprises a wide range of peptides that possess potent ion channel inhibitor activity.

### EXAMPLE 12

### Chimeric SCID Mouse Model for Human Intestine

The study of human gastrointestinal physiology and disease is complicated by the relative inaccessibility of this organ for investigation, as well as a range of ethical considerations. In an attempt to study human gastrointestinal disease under more controlled experimental conditions a chimeric scid (severe-combined immunodeficiency) mouse model for human intestine that provided an opportunity for long-term investigations in vivo was established. Intact segments (2-3 cm lengths) of human fetal intestine (10-15 weeks gestational age) were xenotransplanted into sub-cutaneous tunnels on the back of 6-8 week old C.B-17 scid mice, for durations of up to 1 year. Xenograft vascularisation and the presence of all major intestinal epithelial cell lineages are evident within 2 and 10 weeks following transplantation, respectively. Epithelial differentiation was well advanced at this stage, as indicated by enzyme cytochemistry showing brush border alkaline phosphatase, aminopeptidase-N, lactase, -glucosidase and dipeptidylpeptidase IV activities comparable to those measured in the intestine of children. Double-label in situ hybridisation employing biotinylated and dioxigenin-labeled whole human and mouse DNA probes, performed at both light and electron microscopy levels, demonstrated a human origin for the majority of cellular components comprising the xenografts, including the epithelium. Thus a humanized animal model where intestinal xenografts display a morphology and function similar to paediatric bowel was established.

### EXAMPLE 13

### Epithelial Glycosylation Patterns in Human Xenograft Intestine

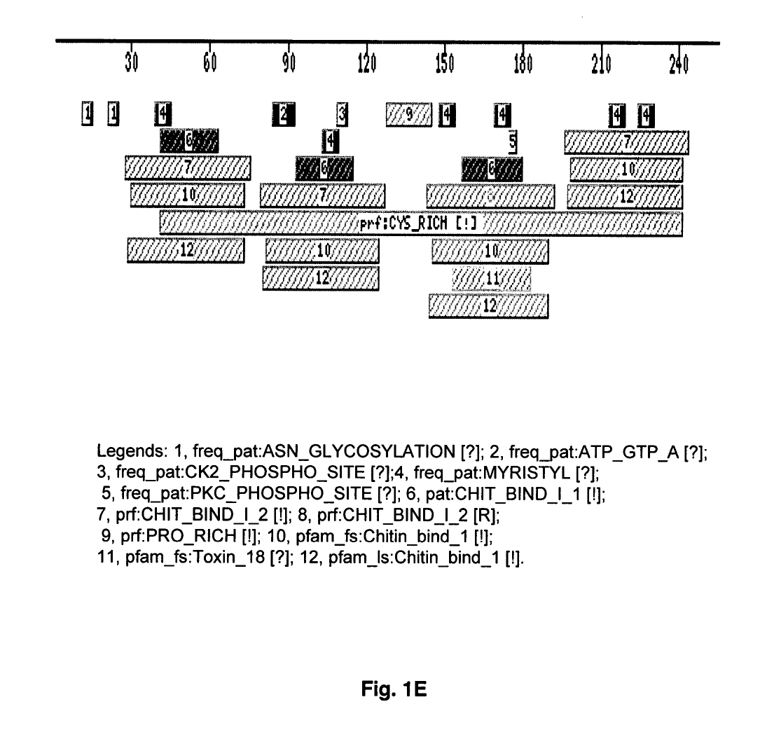

Part of the characterisation process of this model system involved extensive studies of potential carbohydrate receptors in the brush border membrane of non-transformed human intestinal epithelial cells. This was achieved, in part, by measuring glycosylation patterns using a 21-member panel of biotinylated lectins showing different carbohydrate recognition specificities. For these studies levels of lectin binding were quantitatively compared to brush border membranes in paediatric (with and without inflammatory bowel disease (IBD), xenograft and fetal intestine, as well as in adenocarcinoma (HT-29, WiDr, Caco-2, T84, and LIM1864) cell lines. In summary, similar brush border membrane glycosylation patterns in xenograft and pediatric tissues were recorded with the exception of two major groups of carbohydrate specificities. Further investigation of these differential lectin-binding properties in fetal intestine and in adenocarcinoma cell lines demonstrated a similar phenotype to xenografted tissues. Therefore, although xenograft intestine appears morphologically normal it retains two distinct onco-fetal glycosylation patterns. The first group, recognised by the galactose binding lectin Arachis hypogaea, is a well-established onco-fetal glycosylation pattern found in colorectal carcinomas. The second group represents a novel onco-fetal lectin binding pattern (extended oligomers of N-acetyl-glucosamine) which is recognised by the dietary lectin-conotoxin isolated from tomatoes (Lycopersicum esculentum [LEL]) (FIGS. 2A-2B). Because of the large consumption and preferential tumor cell-recognition by this latter group of lectins experiments aimed at characterising the molecular recognition and consequences of tomato lectin-conotoxin binding to adenocarcinoma cell lines in vitro was further investigated.

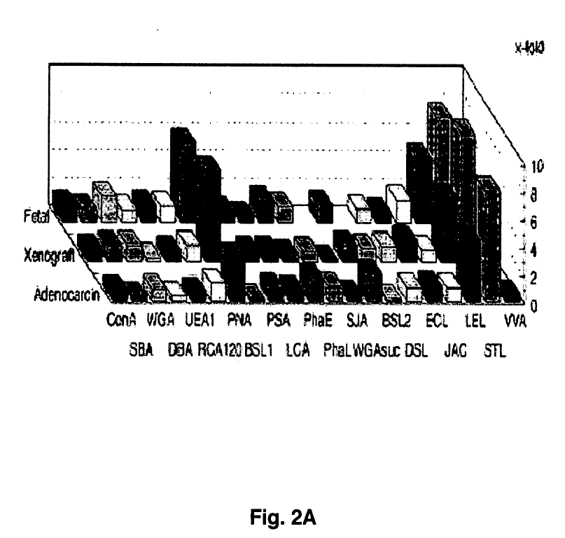

FIG. 3 shows the tomato lectin-conotoxin slot blots of epithelial microvillus membrane preparations. The figure demonstrates high binding of tomato lectin-conotoxin in fetal and xenograft small intestine as compared to pediatric small intestine. Elevated lectin-conotoxin binding was also observed in inflammatory bowel diseases such as Chrohn's disease and ulcerative colitis as compared to normal colon.

### EXAMPLE 14

### Tomato Lectin-Conotoxin Binding to HT29 and Caco-2 Cells

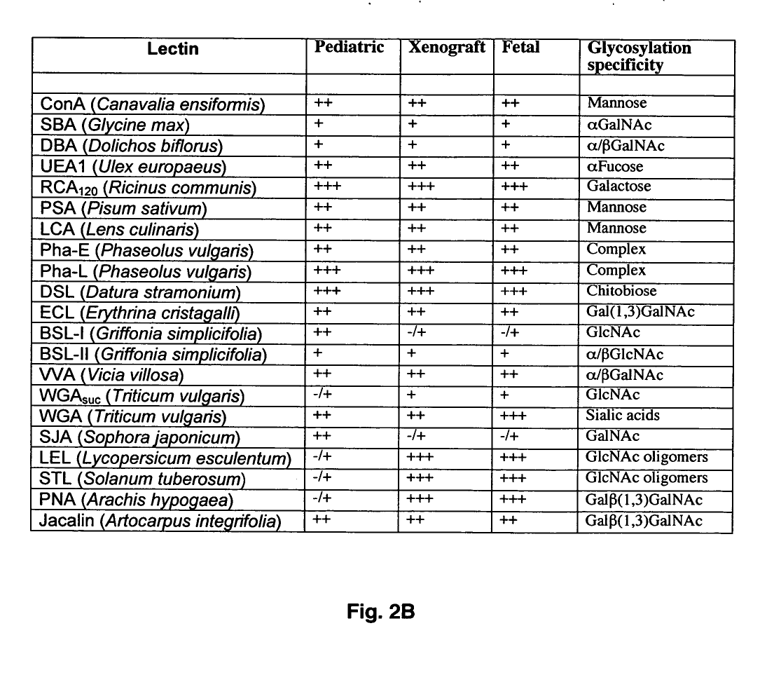

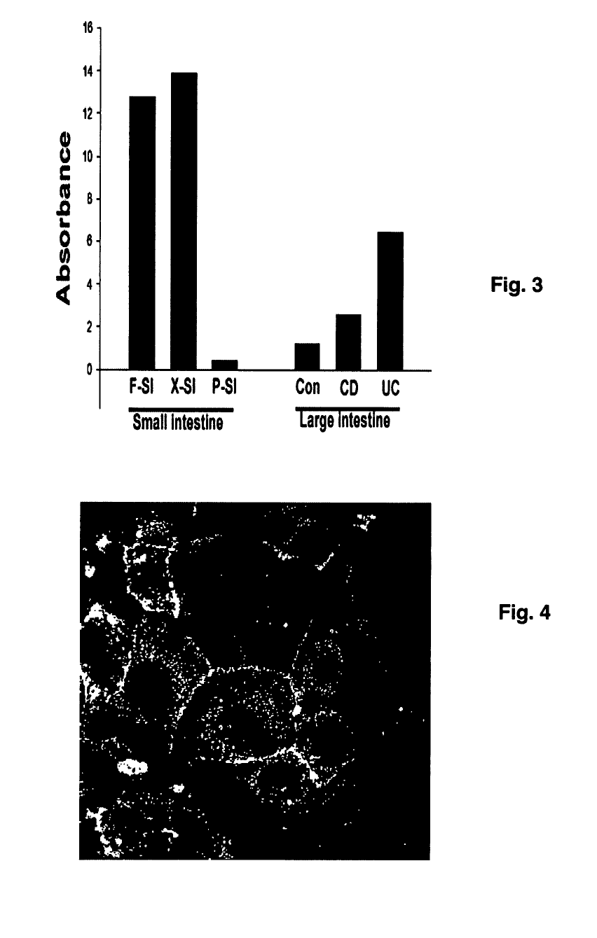

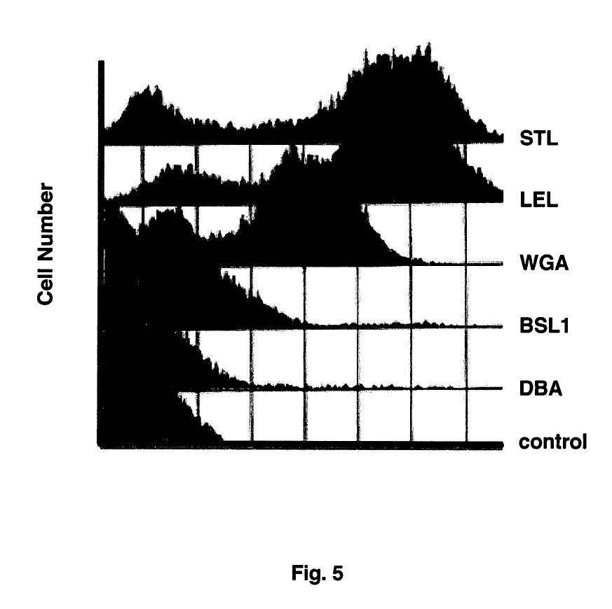

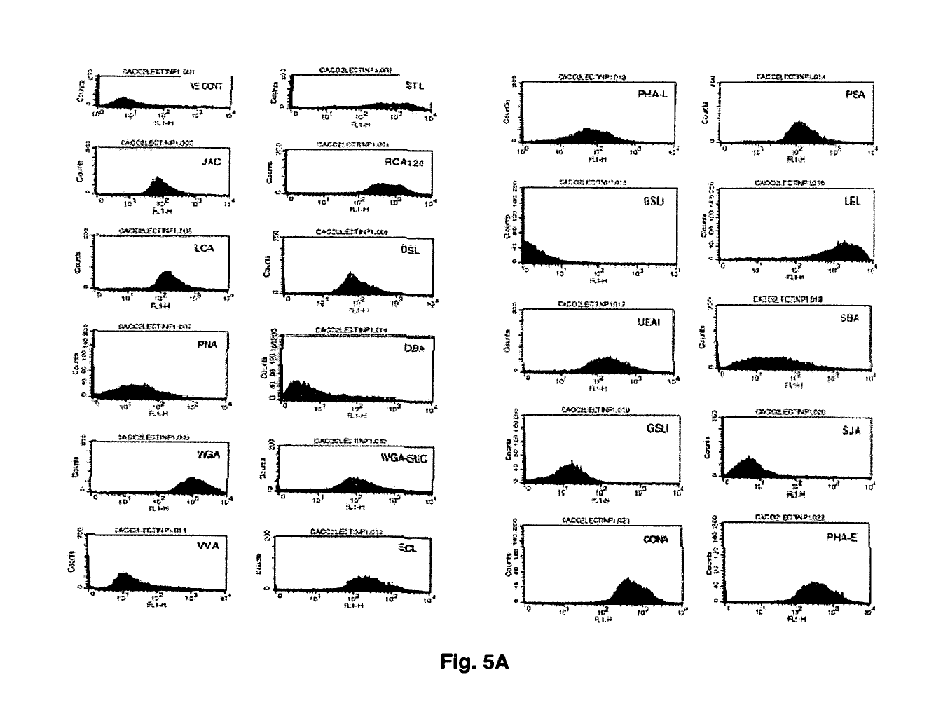

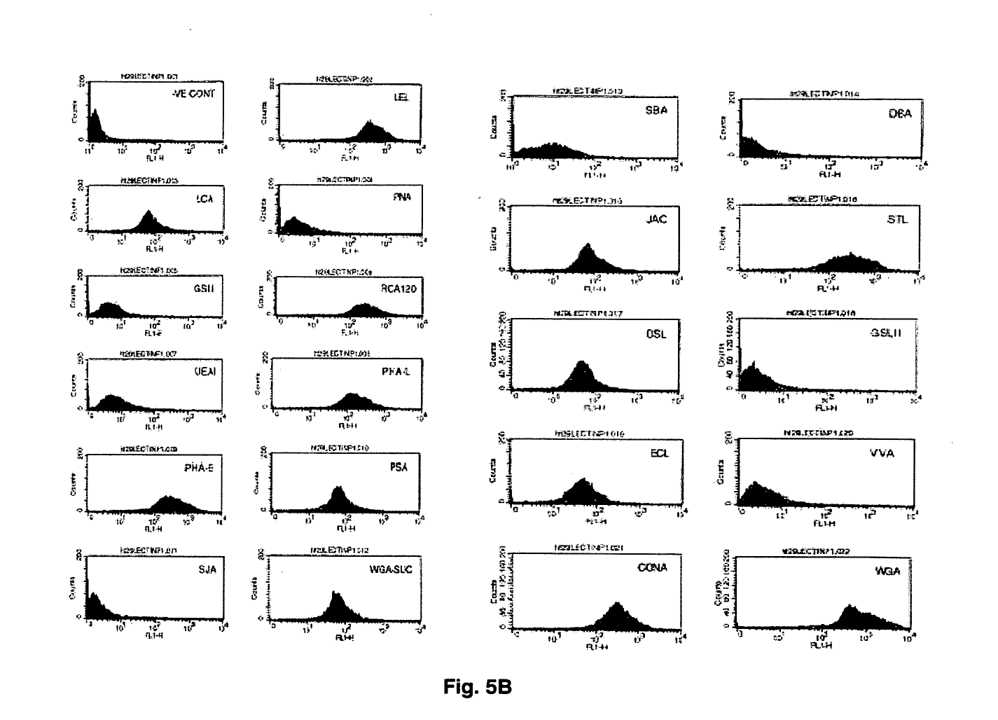

The binding of tomato lectin-conotoxin to Caco-2 cells is illustrated in the confocal micrograph (FIG. 4) that shows FITC-conjugated tomato lectin-conotoxin (10 nM DMEM; green) binding to Caco-2 cells following a 60 min pulse-label at 37° C. The relative capacity of tomato lectin-conotoxin (LEL) binding to Caco-2 and HT29 cells is shown by flow cytometry in FIGS. 5A-5B, where it is compared quantitatively to other lectin binding specificities. Specific binding of tomato lectin-conotoxin to extended oligomers of N-acetyl-glucosamine on the surface of HT29 cells is demonstrated by the competitive inhibition provided by potato lectin (FIG. 6; also specific for oligomers of N-acetyl-glucosamine) and chitin hydrolysate (FIG. 7) that is comprised of oligomers of N-acetyl-glucosamine. In addition, surface plasmon resonance imaging of tomato lectin-conotoxin binding to plasma membrane preparations demonstrated significant real-time binding that was inhibited by chitin hydrolysate (FIG. 8). These studies also demonstrated that the binding affinity of LEL to tumor cell membrane or fetal/xenograft brush border membranes was significantly higher than for normal pediatric or adult intestinal tissues (FIG. 9).

### EXAMPLE 15

### Tomato Lectin-Conotoxin Binding to Adenocarcinoma Cells Inhibits Tyrosine Kinase Activity and Cellular Proliferation

The physiological consequences of tomato lectin-conotoxin binding to HT29 and Caco-2 cells is significant. Within minutes of binding to the cellular apical membrane, tomato lectin-conotoxin inhibits total cellular tyrosine kinase activity (FIGS. 9A-9B). However, preliminary findings indicate that protein kinase C activity is not similarly altered. Protein tyrosine kinases transfers the phosphate of ATP to tyrosine residues of protein substrates, and are critical components of signaling pathways that control cellular proliferation and differentiation.

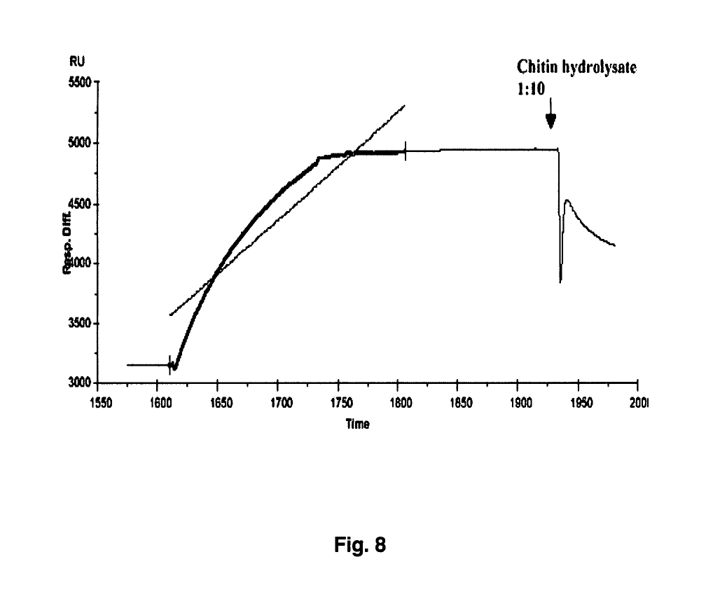

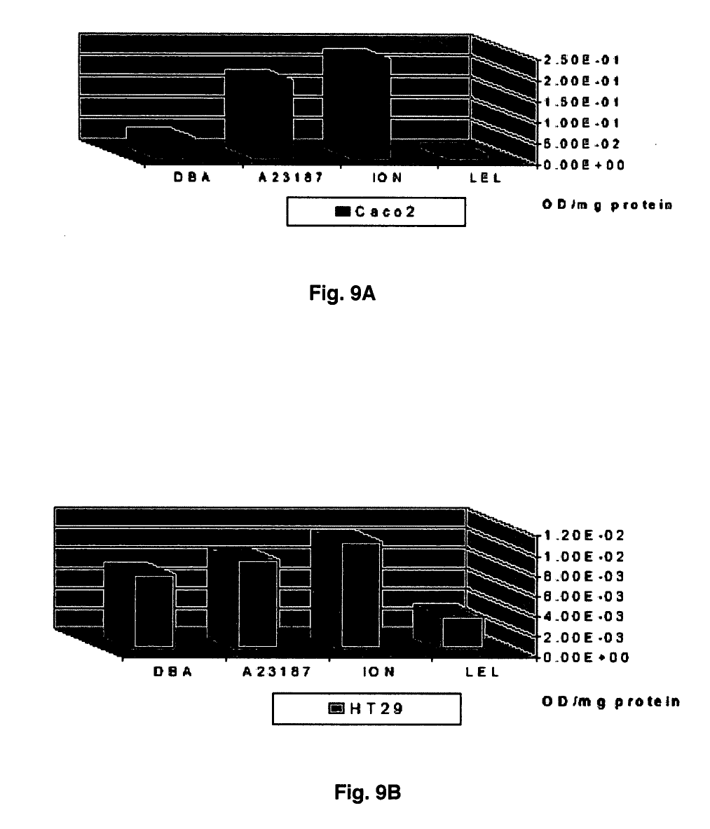

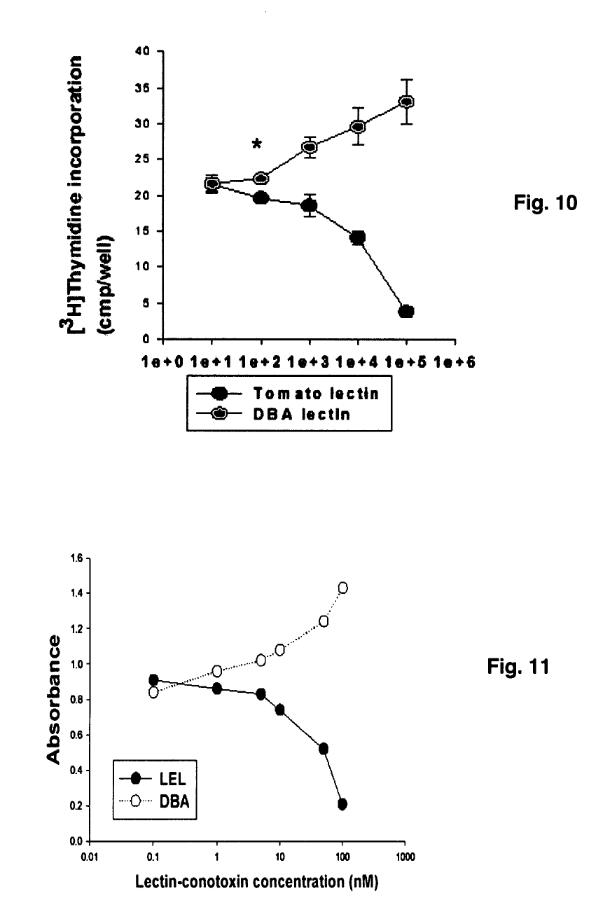

In fact, exposure of HT29 and Caco-2 cells to tomato lectin-conotoxin (10-100 nM range in serum free DMEM) for 48 hours significantly reduced cell numbers (FIGS. 10-11). At higher tomato lectin-conotoxin doses (100 nM) many cells appeared morphologically apoptotic, showing evidence of nuclear fragmentation. This is evident as an increased side scatter profile when analysed using flow cytometry (FIG. 12).

### EXAMPLE 16

### Tomato Lectin-Conotoxin Mediated Induction of Apoptosis in Adenocarcinoma Cells

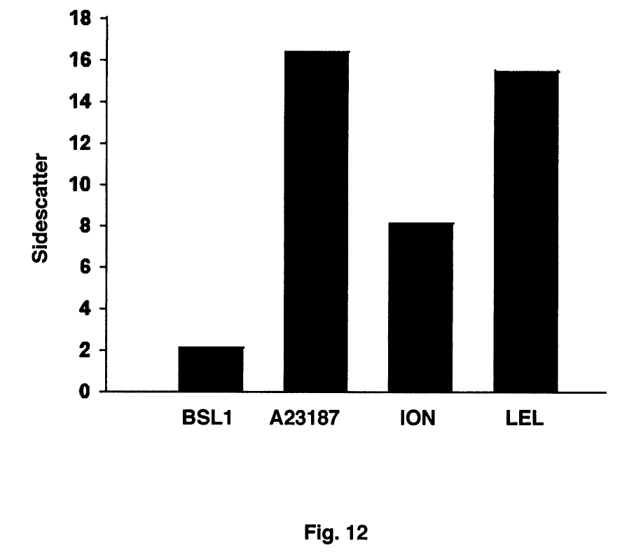

A number of assays were adopted to quantify the extent of tomato lectin-conotoxin induced apoptosis in HT29 and Caco-2 cells; which included measurement of DNA fragmentation, annexin-V binding to membrane phospatidylserine residues, and activation of caspase-3. An early feature of apoptotic cells is a plasma membrane alteration, for example phosphatidylserine translocates from the inner part of the membrane to the outer layer. Under such conditions phoshatidylserine may therefore be detected using annexin-V, a Ca2+ dependent phoshoplipid-binding protein with a high affinity for phosphatidylserine. A FACS profile demonstrating induction of apotosis in HT29 cells 24 hours after treatment with TNFα or Fas ligand (20 ng/ml and 1 μg/ml; both with 100 U/ml IFN-γ) and tomato lectin-conotoxin (100 nM) is shown in FIGS. 13A-13C.

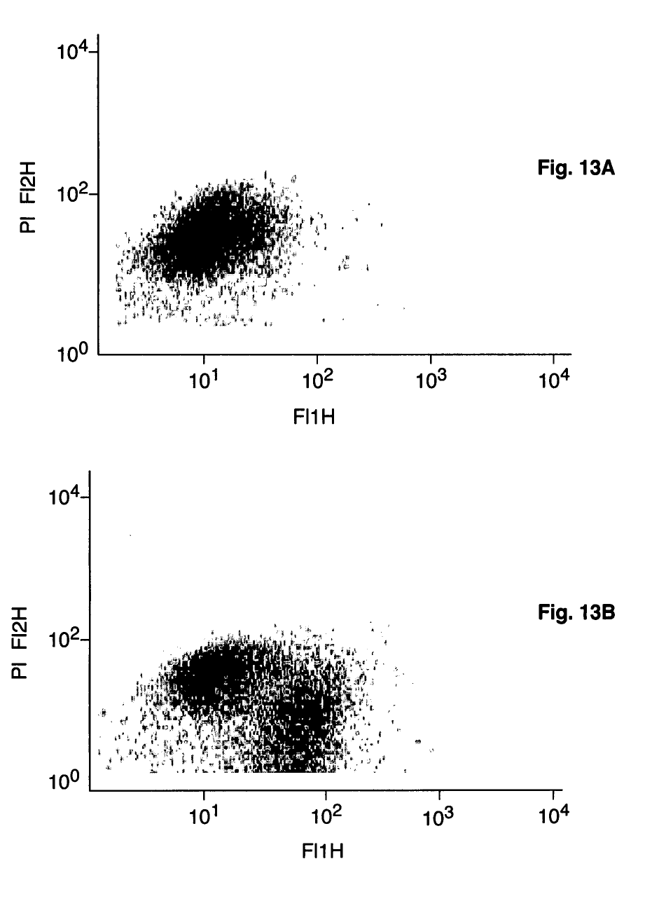

Another early indicator of apoptosis is the switch on of several ‘death genes’, for example, activation of members of the interleukin-1β converting enzyme (ICE) family. One of these cysteine proteases, CPP32 or caspase-3, plays a direct role in the proteolytic digestion of cellular proteins responsible for progression to apoptosis. Its measurement may therefore be used as an indicator of apoptotic activity in complex cell populations. Levels of CPP32 activity in HT29 and Caco-2 adenocarcinoma cells are shown in FIG. 14 following treatment with Fas ligand, TNFα or tomato lectin-conotoxin for 24 hours. As for the Annexin-V profiles, Fas ligand and TNFα induced a marked increase in CCP32 activity in HT29 cells. This feature was less apparent in Caco-2 cells that do not express high levels of suitable Fas or TNFα receptors. A significant elevation was also observed in Caco-2 cells incubated with tomato lectin-conotoxin (100 nM for 24 hours).

### EXAMPLE 16

### Tomato Lectin-Conotoxin Mediated Induction of Cellular Differentiation of Adenocarcinoma Cells

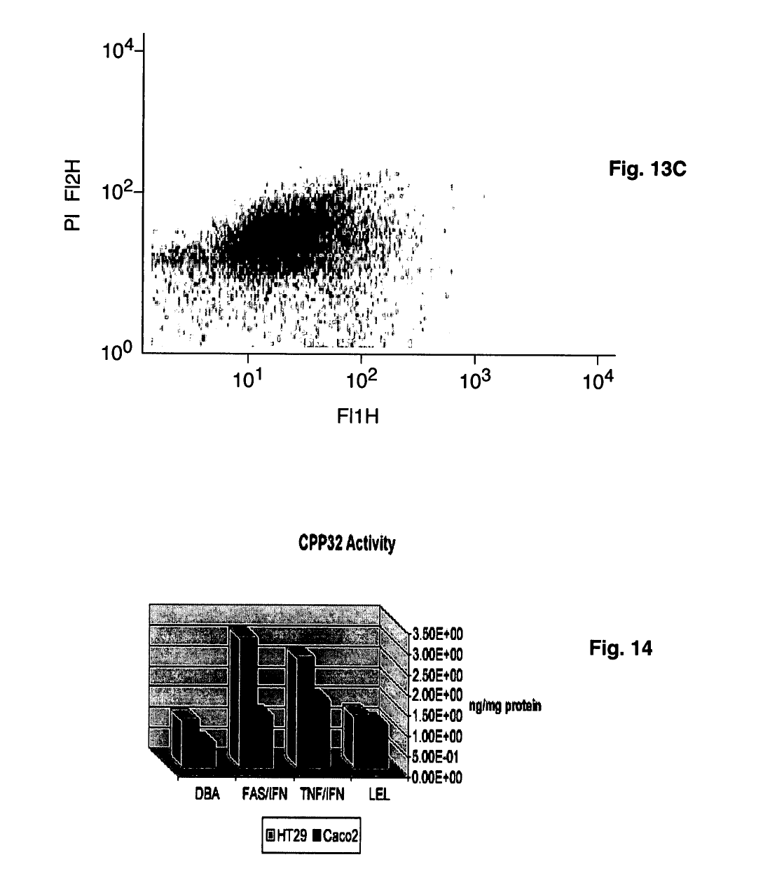

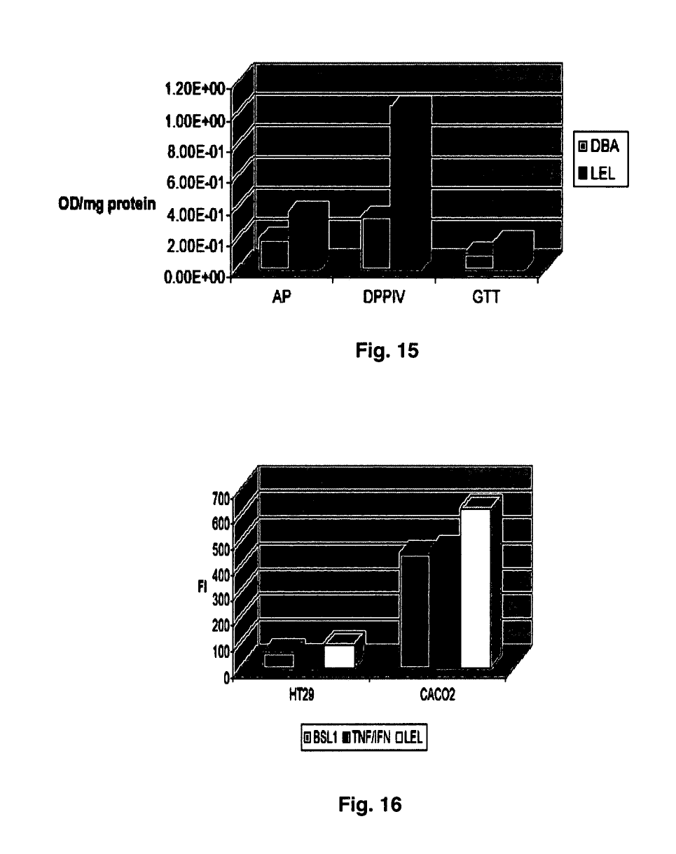

Measurement of brush border alkaline phosphatase and dipeptidyl peptidase IV activity, both of which are indicators of cellular differentiation in intestinal epithelial cells, demonstrated elevated values following incubation with 100 nM tomato lectin-conotoxin for 24 hours (FIG. 15). This correlated with an increased immunoreactivity as assessed by flow cytometry (FIG. 16).

### EXAMPLE 17

### Association of Tomato Lectin-Conotoxin with Tumor Rejection Antigen gp96

Tomato lectin-conotoxin binds to several cellular glycoproteins, although association with a 96 kDa glycoprotein species is potentially important in regulating tumor cell proliferation and programmed cell-death. Isolation of this glycoprotein and identification using mass spectrometry has identified this as the tumour rejection antigen, gp96. gp96 is an inducible homologue of the heat shock protein 90 (HSP90). Overall the relationship between gp96 structure and function is poorly understood, although it is believed to chaperone cellular peptides providing proper folding of nascent polypeptides and protecting polypeptides from denaturing during cellular stress.

gp96 is greatly up regulated during malignant transformation, where it is located within the endoplasmic reticulum and in the plasma membrane. This glycoprotein contains 5 potential N-glycosylation sites that are differentially glycosylated in malignant cells, allowing potent binding of tomato lectin-conotoxins to cancer cells (10). In these studies, however, it was demonstrated that gp96 (and HSP90/70 which may also be targets for tomato lectin-conotoxin) may chaperone important regulators of tumor cell proliferation and survival. Polypeptides identified thus far include several zinc-finger binding proteins e.g. ZNF225 which acts an important regulatory transcription factor in tumor cells. Other examples of important regulators found to associate with this complex include proliferation cell nuclear antigen (PCNA) and B23 nucleophosmin (which interacts with p53). In view of these findings, it is conceivable that dysfunction of gp96 function (or indeed other HSP) due to an interaction with tomato lectin-conotoxin could result in an inhibition of tumor cell proliferation and death.

Tomato lectin-conotoxin is a chimeric protein consisting of homologous chitin-binding modules, separated by an extension-like linker and a conotoxin-like motif (FIGS. 1A-1B). The extensin-like repeats are hydroxyproline-rich glycoprotein domains that have been implicated in pathogen host defense and in the bioassembly of extracellular matrix (9). Several proposed functions have been suggested for the extensins, including adhesion and cross-linking of proteins and binding hydrophobic ligands. A conotoxin-like domain is also contained within this molecule, and may have important biological/anti-tumour activity. It is contemplated that these independent chimeric functions may act in concert to inactivate molecules such as gp96 on tumor cells, most likely by inhibiting gp96 mediated protein conformation/refolding resulting in a depletion of oncogenic kinases through proteosomal degradation of immature protein. This notion is supported by the fact that tyrosine kinase signaling is significantly suppressed in tomato lectin-conotoxin treated tumor cells. The unique glycosylation pattern of pg96 can promote initial binding to dietary tomato lectin-conotoxin. An additional extensin-like domain function of the tomato lectin-conotoxin can then subsequently inactivate gp96 function, resulting in altered cellular physiology and death (FIGS. 17A-17B).

### EXAMPLE 18

### Association of Tomato Lectin-Conotoxin with Membrane Ion Channels

Tomato lectin-conotoxin binds to several membrane glycoproteins that could subsequently promote association and inhibition of ion channel activity that is important in regulating tumor cell proliferation and programmed cell-death. An initial interaction of tomato lectin-conotoxin with membrane gp96 and/or another glycoprotein species including ion channels themselves may promote the tomato lectin-conotoxin motif to directly inhibit ion channel activity. L-type calcium channel activity is greatly up regulated during malignant transformation, where it is located within the endoplasmic reticulum and in the plasma membrane (11, 12). It is conceivable that dysfunction of ion channel function due to an interaction with tomato lectin-conotoxin could result in an inhibition of tumor cell proliferation and death (FIGS. 18A-18B).

The significant feature of the tomato lectin-conotoxin protein is that it contains a domain that is similar to the ω-conotoxin domain found in cone snails (13). Small synthetic peptides having this domain have been shown to be toxic mainly by exerting an effect on calcium channels. Based on the prediction that tomato lectin-conotoxin may possess potent ion channel inhibitory activity, other important biological functions may be attributed to the consumption and or use of tomato lectin-conotoxins, for example, in the treatment of chronic pain, inflammation and/or as an anti-microbial. FIG. 19 shows inhibition of cellular calcium entry in mEGC glial cells by tomato lectin-conotoxin (LEL) compared with DBA control lectin at a similar concentration. Extracellular calcium entry into the mEGC cells was mediated by stimulation of voltage-gated channels by the addition of 60 mM KCl. Real-time recording of [Ca²⁺]ᵢ was performed in single cells.

In brief, cells grown on glass coverslips (Carolina Biological) were washed with a physiological medium (KRH) containing in mmol/liter: NaCl 125; KCL 5; KH₂PO₄ and MgSO₄ 1.2; CaCl₂ 2; glucose 6; HEPES-NaOH buffer 25, pH 7.4, then loaded with 2 μM fura-2 AM with 0.05% pluronic F-127 for 50 min. at 25° C. to minimize dye compartmentalization. Loaded cells were washed three times with KRH and incubated for 60 min. at 25° C. in the dark with KRH 0.1% BSA. Loaded cells attached to coverslips were mounted on a Leiden Cover Slip Dish and placed in an Open Perfusion Micro-Incubator (Medical Systems Corp. New York) covered with 3 ml KRH with 0.1% BSA. The calcium variations at the single-cell level were monitored using a Nikon Diaphot inverted microscope (Garden City, N.Y.), equipped with a Nikon 40× (1.3 N.A.) oil immersion objective, coupled to a dual monochrometer system via a fiberoptic cable (Photon Technology International (PTI), South Brunswick, N.J., U.S.A). Fura-2 intracellular fluorescence was measured at an emission wavelength of 510 nm by alternating the excitation wavelength between 340 and 380 nm. Full ratio images were obtained at 1 image per 1.5 seconds. Images were processed using ImageMaster software (PTI).

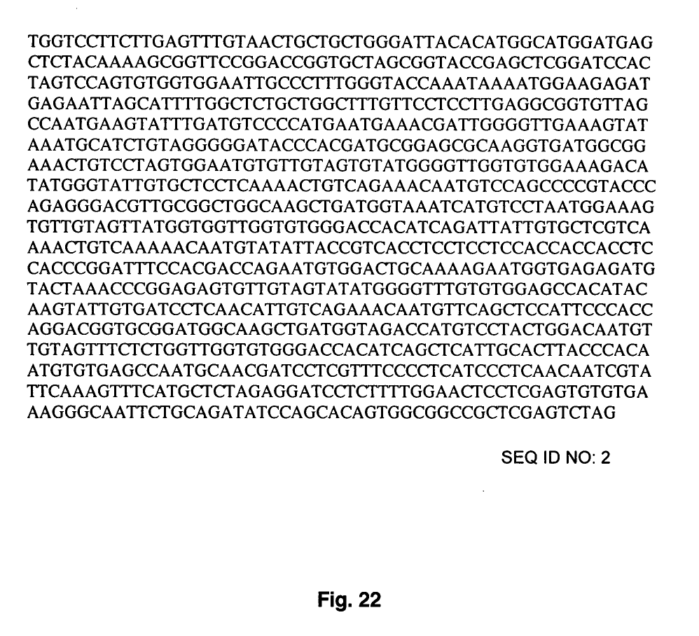

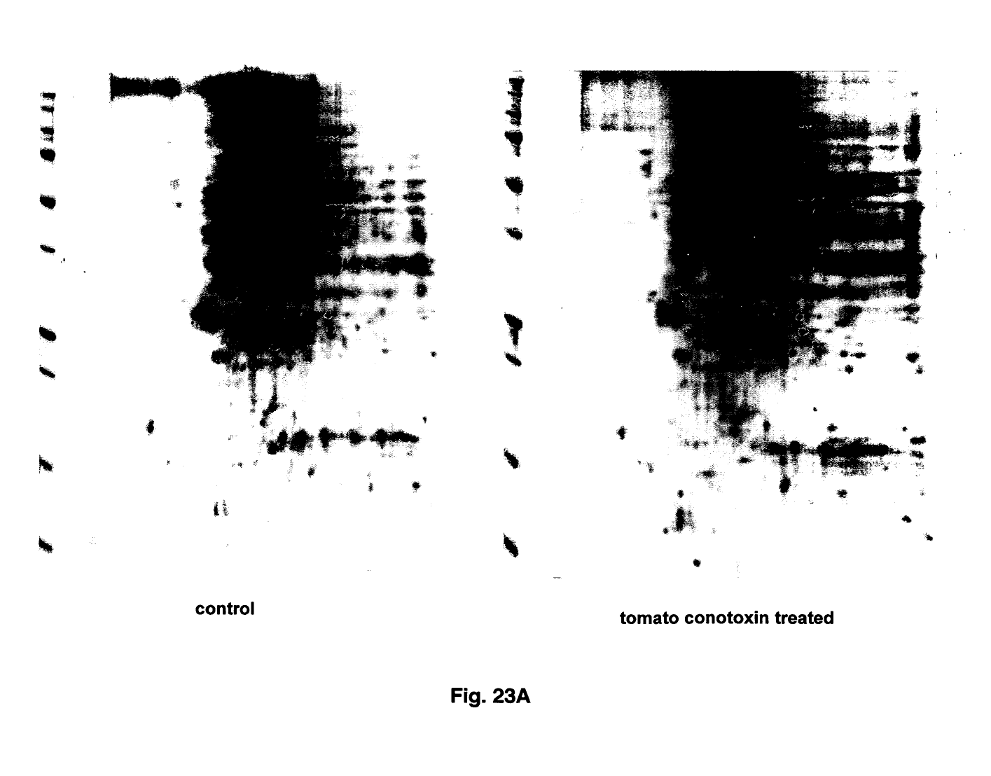

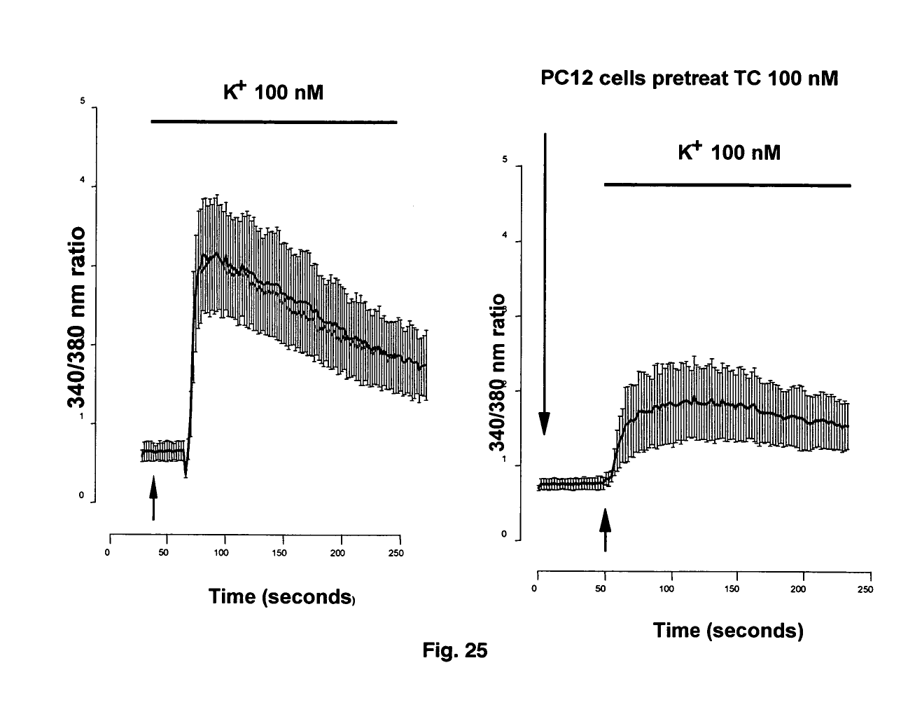

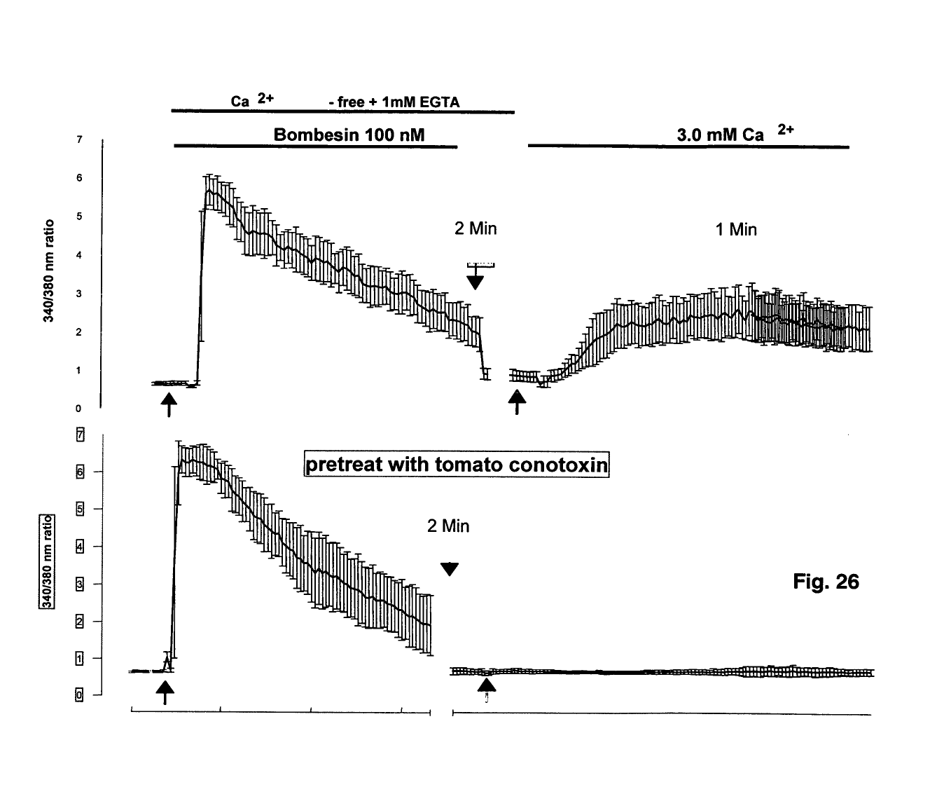

The tomato lectin-conotoxin, LEL, was found to inhibit the intracellular calcium clearance in HEK cells in response to gastrin (FIGS. 21A-21B). FIG. 21C shows that LEL was also found to inhibit intracellular release of calcium by carbachol. Carbachol, a derivative of acetylcholine, is known to increase intracellular levels of calcium. These results indicate that LEL inhibits calcium channel function and is responsible for slow release of intracellular calcium on stimulation of HEK cells with either gastrin or carbachol. FIG. 22 shows the nucleotide sequence encoding LEL. LEL also inhibits voltage-gated calcium channels on PC12 neuronal cells (FIG. 25) and store-operated and TRP channel activity in PC3 prostate cancer cells (FIG. 26). This signaling pathway is important in tumor cells and may explain cytotoxicity.

### EXAMPLE 19

### In Vitro Expression of Tomato Lectin-Conotoxin Gene in Tumor Cells

FIG. 20 shows the expression of the tomato lectin-contoxin in Caco-2 cells. The expression vector used to clone the LEL gene is the Vivid Colors™ pcDNA™ 6.2/EmGFP-Bsd/V5-DEST vector from Invitrogen (Carlsbad, Calif.). This clearly demonstrates the death of cells transfected with the sense constructs of the LEL gene. These results indicate that direct expression of the tomato lectin-conotoxin in cancer cells can be used to eliminate the cancer cells.

The conotoxin motif containing peptide of the tomato-lectin can also be expressed in target cells instead of the entire protein to treat pain/inflammation or to combat a microbial infection. The conotoxin containing nucleotide sequence from LEL (ATGGGTGAGAGATGTACTAAACCCGGAGAG TGTTGTAGTATATGGGGTTTGTGTGGAGCCACATACAAGTATTGTGATCCTCA, SEQ ID NO: 4) or any other related conotoxin nucleotide sequence (ATGTG CAAGGGCAAGGGCGCCAAGTGCTCCCGCCTCATGTACGACTGCTGCACCGGCTCCT GCCGCTCCGGCAAGTGCGGC SEQ ID NO: 5) is cloned into a suitable expression vector such as the Vivid Colors™ pcDNA™ 6.2/EmGFP-Bsd/V5-DEST vector from Invitrogen (Carlsbad, Calif.). This expression vector is delivered to target cells, whereby the vector expresses the conotoxin peptide.

The following references were cited herein:
1. Torres-Pinedo R (1983) J Paed Gastro Nutr 2; 588-594.
2. Hakomori S I, (1989) Adv Canc Res 52; 257-331.
3. Muramatsu T, (1993) Glycobiol 3; 294-296.
4. Dennis et al. (1987) Science 236; 582-585.
5. Dennis J W (1986) Canc Res 46; 5131-5136.
6. Holmes et al. (1987). J Biol Chem 262; 15649-15658.
7. Leathem A J and Brooks S A, (1987) Lancet 1; 1054-1056.
8. Kilpatric et al., (1985). FEBS Lett 185; 299-304.
9. Peumans et al. (2003). Biochem J 376; 717-724.
10. Suriano et al., (2005). Cancer Res 65; 6466-75.
11. Wang et al., (2000). Am J. Path. 157; 1549-1562.
12. Schindelholz B, Reber B F X (2000). Eur J Neuroscience 12; 194-204.
13. Norton R S, Pallaghy P K (1998). Toxicon 36; 1573-1583.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A carbohydrate motif comprising extended oligomers of N-acetylglucosamine units.

2. The carbohydrate motif of claim 1, wherein the motif is present in glycoproteins of cancer cells.

3. The carbohydrate motif of claim 1, wherein the cancer cells are adenocarcinoma, melanoma, lymphoma or glioma cells.

4. The carbohydrate motif of claim 3, wherein the adenocarcinoma is intestinal adenocarcinoma, prostate adenocarcinoma or renal adenocarcinoma.

5. The carbohydrate motif of claim 1, wherein the motif binds the tomato fruit lectin-conotoxin obtained from Lycopersicum esculentum.

6. A pharmaceutical composition comprising a lectin-conotoxin that binds the carbohydrate motif of claim 1 and a pharmaceutically acceptable vehicle.

7. The pharmaceutical composition of claim 6, wherein the lectin-contoxin is purified from a natural source or is a bioengineered molecule.

8. The pharmaceutical composition of claim 6, wherein the lectin-conotoxin comprises one or more chitin binding domain(s), an extensin domain and a conotoxin domain.

9. The pharmaceutical composition of claim 6, wherein the lectin-conotoxin comprises two chitin binding domains separated by an extensin domain and a conotoxin domain.

10. The pharmaceutical composition of claim 6, wherein the lectin-conotoxin comprises the amino acid sequence of SEQ ID NO: 1

11. The pharmaceutical composition of claim 6, wherein the lectin-conotoxin is from the fruit of Lycopersicum esculentum.

12. The pharmaceutical composition of claim 6, wherein the lectin-conotoxin is effective to suppress tyrosine kinase activity or ion channel activity in a cell.

13. The pharmaceutical composition of claim 12, wherein the lectin-conotoxin binds ion channels in a cell.

14. The pharmaceutical composition of claim 13, wherein the ion-channels bound are the voltage-gated, storage operated and transient receptor potential calcium channels.

15. The pharmaceutical composition of claim 6, wherein the lectin-conotoxin binds GP96 heat shock protein.

16. The pharmaceutical composition of claim 15, wherein the binding is effective to suppress GP96.

17. The pharmaceutical composition of claim 16, wherein the GP96 suppression is effective to induce an endoplasmic reticulum stress response.

18. A method to retard growth and proliferation of cancer cells, comprising:
contacting said cancer cells with the pharmaceutical composition of claim 6.

19. The method of claim 18, wherein said cancer cells are adenocarcinoma, lymphoma, melanoma or glioma cells.

20. The method of claim 18, wherein said adenocarcinoma is intestinal adenocarcinoma, prostate adenocarciona or renal adenocarcinoma.

21. A method of treating a pathophysiological state in an individual comprising:
administering the lectin conotoxin comprising the pharmaceutical composition of claim 6 to the individual.

22. The method of claim 21, wherein the pathophysiological state is one or more of a cancer, chronic pain, inflammation or a microbial infection.

23. The method of claim 22, wherein the cancer is intestinal adenocarcinoma, prostate adenocarcinoma, renal adenocarcinoma, melanoma, lymphoma or glioma.

24. The method of claim 21, further comprising:
administering one or more of an anti cancer agent, an analgesic/anti-inflammatory agent or an antimicrobial agent to the individual.

25. The method of claim 24, wherein the anticancer agent is a chemotherapeutic agent or a radiotherapeutic agent.

26. The method of claim 24, wherein the analgesic/anti-inflammatory agent is aspirin, paracetamol, ibuprofen, ketoprofen, naproxen sodium, diflunisal, indomethacin, sulindac, or corticosteroids.

27. The method of claim 24, wherein the anitimicrobial agent is an antibiotic or an antifungal agent.

28. A genetically modified plant, wherein the plant overexpresses a lectin-conotoxin comprising the sequence of SEQ ID No: 1.

29. The Seeds, fruits, progeny and hybrids of the tomato plant of claim 28.

30. The plant of claim 28, wherein the plant is a tomato plant.

31. An expression vector comprising the nucleic acid sequence encoding a lectin-conotoxin comprising the sequence of SEQ ID NO: 1 and regulatory elements required to express the lectin-conotoxin.

32. The expression vector of claim 31, wherein the nucleic acid sequence comprises the sequence of SEQ ID NO: 2.

33. A genetically modified food product, wherein the food product comprises excessive amounts of a lectin-conotoxin comprising the sequence of SEQ ID NO: 1.

34. The food product of claim 33, wherein the food product is seeds, fruits, vegetables, eggs, milk, or meat.

35. A conotoxin motif having the amino acid sequence of SEQ ID NO:3.

36. DNA encoding a protein, wherein the protein has a conotoxin motif and wherein the DNA is:
(a) isolated and purified DNA consisting of the sequence of SEQ ID NO: 4.
(b) isolated and purified DNA which hybridizes at high stringency conditions to the antisense complement of the isolated DNA of (a) above; or
(c) isolated and purified DNA differing from the isolated DNAs of (a) and (b) above in codon sequence due to the degeneracy of the genetic code.

37. An expression vector capable of expressing the DNA of claim 36, wherein the vector comprises regulatory elements necessary for expression of the DNA in a cell.
